(19) United States
(12) Patent Application Publication
(10) Pub. No.: US 2026/0027082 A1
(43) Pub. Date: Jan. 29, 2026

(54) METHODS FOR TREATING TREATMENT RESISTANT DEPRESSION

(71) Applicant: Atai Therapeutics, Inc., New York, NY (US)

(72) Inventors: Kevin CRAIG, London (GB); Robert W. BUSBY, Hingham, MA (US)

(21) Appl. No.: 19/284,159

(22) Filed: Jul. 29, 2025

Related U.S. Application Data
(60) Provisional application No. 63/676,765, filed on Jul. 29, 2024.

(51) Int. Cl. A61K 31/4045; A61K 9/00; A61P 25/24

(52) U.S. Cl. CPC A61K 31/4045; A61K 9/006; A61P 25/24

(57) ABSTRACT

The present disclosure provides methods and dosing regimens for the treatment of depression (e.g., treatment resistant depression) with a mucoadhesive composition comprising N, N-dimethyltryptamine (DMT) or a pharmaceutically acceptable salt thereof.

### CROSS-REFERENCE TO RELATED APPLICATIONS

This application claims the benefit of and priority to U.S. Provisional Application No. 63/676,765, filed Jul. 29, 2024, the contents of which are hereby incorporated by reference in their entirety for all purposes.

### BACKGROUND

Depression is one of the most common mental illnesses, affecting more than 264 million people worldwide. It is characterized by depression and a marked decrease in interest or joy in the activity. Other symptoms include significant weight loss or weight gain, loss or increase in appetite, insomnia or hypersomnia, psychomotor hypersomnia or delay, fatigue or loss of energy, feelings of worthlessness or excessive or inappropriate guilt, poor thinking or concentration or lack of determination, thinking about recurring deaths, suicidal ideation, or attempted suicide.

Current treatments for depression often consist of a combination of psychotherapy and one or more daily medications that regulate neurotransmitters such as dopamine, serotonin, and norepinephrine. These agents often take weeks or months to achieve their full effect, during which time individuals continue to suffer from symptoms, self-harm, and personal and professional life.

N, N-dimethyltryptamine (DMT) holds therapeutic value as a psychedelic, with efficacy trials ongoing to assess the effect of DMT or DMT fumarate administered intravenously to subjects with major depressive disorder (“MDD”). However, although the intrinsic properties of DMT make it an attractive possible medication, especially for neurological diseases and conditions, current therapeutic compositions and modes of administration complicate treatment and may not provide optimal therapeutic results. For example, when smoked or delivered intravenously, DMT has a very fast onset of action and a short duration of effect, which presents a challenge to determine a suitable administration regimen with appropriate dosage and frequency of administration of DMT to provide effective therapy for neurological diseases and conditions. This is especially true for neurological diseases and conditions which would benefit from the presence of therapeutic blood levels of DMT over a more extended period of time following administration.

Therefore, there remains a need for methods of treating depression, more particularly treatment resistant depression, using readily administrable medications of DMT.

### SUMMARY

In aspects, the disclosure provides methods of treating treatment resistant depression (TRD) in a patient in need thereof, the method comprising administering a mucoadhesive composition comprising N, N-dimethyltryptamine (DMT) or a pharmaceutically acceptable salt thereof, wherein the administration provides a DMT Tₘₐₓ of about 0.3 h to about 2 h and a DMT Cₘₐₓ of about 5 ng/mL to about 80 ng/mL following administration.

In aspects, the disclosure provides methods of treating TRD in a patient in need thereof, the method comprising administering a mucoadhesive composition comprising N, N-dimethyltryptamine (DMT) or a pharmaceutically acceptable salt thereof, wherein the administration provides a DMT Tₘₐₓ of about 0.3 h to about 2 h and a DMT AUCₗₐₛₜ of about 3 ng·h/mL to about 70 ng·h/mL following administration.

In embodiments, about 20 mg to about 200 mg of DMT free base or an equivalent dose of the pharmaceutically acceptable salt thereof is administered to the patient. In embodiments, the composition is administered buccally. In embodiments, the composition is administered through a transmucosal application. In embodiments, the composition is administered once every about 2 weeks to about 6 months.

In embodiments, the administration provides a reduction in the patient's Montgomery-Asberg Depression Rating Scale (MADRS) total score by at least about 50% compared to prior to the administration.

In aspects, the present disclosure provides methods of treating TRD by administering DMT or a pharmaceutically acceptable salt thereof to a patient in need thereof, the method comprising: a) administering to the patient an induction regimen comprising a DMT induction dose; and b) thereafter administering to the patient a maintenance regimen comprising a DMT maintenance dose.

In aspects, the present disclosure provide methods of treating TRD by administering DMT or a pharmaceutically acceptable salt thereof to a patient in need thereof, the method comprising: a) administering to the patient an induction regimen comprising a DMT induction dose, wherein the induction regimen provides remission of the patient's TRD; and b) thereafter administering to the patient a maintenance regimen comprising a DMT maintenance dose.

In embodiments, the patient is at least 18 years old. In embodiments, the patient has a diagnosis of moderate or severe major depressive disorder (MDD) without psychotic features and is currently experiencing a major depressive episode. In embodiments, the patient has the onset of the first episode of MDD before age 55. In embodiments, the patient has not responded to an adequate dose and duration of at least two and no more than five antidepressant medications for the current depressive episode. In embodiments, the patient has a MADRS total score of at least about 20 points prior to the administration.

Other aspects and embodiments will be apparent to one skilled in the art in light of the detailed description and Examples that follow.

### BRIEF DESCRIPTION OF THE DRAWINGS

FIG. 1 shows the study design for Part A of the Phase 1 study.

FIGS. 2A-2B show the Cₘₐₓ (FIG. 2A) and AUCₗₐₛₜ (FIG. 2B) box plots for subjects in the DMT IV dose group or in the DMT buccal thin film (BTF) dose groups.

FIG. 3 shows DMT plasma concentration over time for subjects in the DMT IV dose group or in the DMT BTF dose groups.

FIG. 4 shows SIRS overtime for subjects in the DMT IV dose group or in the DMT BTF dose groups.

FIG. 5 shows the study design for the Phase 2 study.

### DETAILED DESCRIPTION

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5.

The terms “administer,” “administering” or “administration” as used herein refer to administering a compound or pharmaceutically acceptable salt of the compound or a composition or formulation comprising the compound or pharmaceutically acceptable salt of the compound to a patient.

The terms “subject” “individual” and “patient” are used interchangeably herein and refers to a human.

The terms “treating” as used herein with regard to a patient, refers to improving at least one symptom of the patient's disorder. Treating can be improving, or at least partially ameliorating a disorder or an associated symptom of a disorder.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. Salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result.

The term “N, N-Dimethyltryptamine” or “DMT” as used herein means a compound of formula (I):

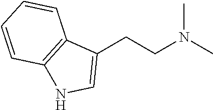

In embodiments, the DMT administered in the methods of present disclosure is a DMT ester or a DMT prodrug. In embodiments, the DMT administered in the methods of present disclosure is a pharmaceutically acceptable salt of DMT.

### Compositions

In aspects, the present disclosure provides mucoadhesive compositions comprising N, N-dimethyltryptamine (DMT) or a pharmaceutically acceptable salt thereof. DMT-containing mucoadhesive compositions and methods of their manufacture are disclosed in U.S. Pat. Nos. 11,602,521 and 12,128,027; and International Publication No. WO 2025/076151, the contents of each of which are hereby incorporated by reference in their entireties for all purposes.

In embodiments, the mucoadhesive composition of the present disclosure comprises a pharmaceutically effective amount of an active DMT agent, the active DMT agent comprising DMT or a pharmaceutically acceptable salt thereof, wherein the pharmaceutically effective amount of the active DMT agent is greater than about 27%, including from about 28%, about 29%, or about 30% to about 60% by weight of the mucoadhesive composition; and a polymeric carrier matrix, the polymeric carrier matrix stabilizing the DMT agent in an amorphous form within the mucoadhesive composition, wherein the polymeric carrier matrix is present in the mucoadhesive therapeutic composition in an amount of about 15% to about 50% by weight of the mucoadhesive composition. In embodiments, the stabilization of the DMT agent in an amorphous form is characterized by a powder x-ray diffractogram free of any discernable peaks that are attributable to the active DMT agent.

In aspects, the composition of the present disclosure uses a polymeric carrier matrix that is suitable for transmucosal delivery. In embodiments, the polymeric carrier matrix comprises carboxymethyl cellulose (CMC), hydroxypropyl cellulose (HPC), and a copolymer of N-vinyl-2-pyrrolidone and vinyl acetate. In embodiments, the polymeric carrier matrix comprises an HPC, wherein the HPC comprises HPC-L, HPC-GXF, and HPC-SSL. In embodiments, the transmucosal delivery is buccal, sublingual, gingival, on the tongue, nasal, vaginal, or a rectal delivery. In embodiments, the film is placed in the oral cavity with the backing layer facing away from the patient's skin (i.e., on the side of the film opposite that which is put into contact with the patient's skin).

In embodiments, the composition of the present disclosure maintains the DMT within an oral thin film (e.g., buccal film) in an amorphous state. In embodiments, the amorphous state of the DMT can be confirmed by a differential scanning calorimetry (DSC) spectrum lacking a sharp melting endotherm of crystalline DMT and/or lacking an indication of phase change. In embodiments, the mucoadhesive compositions comprise DMT into an oral thin film in the range of greater than about 27%, including from about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, to about 60% by weight of the mucoadhesive composition.

In embodiments, the mucoadhesive composition comprises an oral thin film comprising a polymeric carrier matrix in the range of about 15% to about 50%, including from about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, by weight of the mucoadhesive composition.

In embodiments, the mucoadhesive composition comprises a buffering agent, which is present in a concentration of about 0.1% to about 10% by weight of the mucoadhesive composition. In embodiments, the buffering agent comprises one or more of citric acid, tartaric acid, fumaric acid, succinic acid, malic acid, sodium citrate, sodium tartrate, sodium succinate, sodium fumarate, or any combination thereof.

In embodiments, the mucoadhesive composition comprises an antioxidant and permeation enhancer, which together is present in an amount of about 2% to about 10% by weight, preferably about 4% to about 8% by weight, and more preferably about 5% to about 6% by weight. In embodiments, the antioxidant comprises glutathione and is present in an amount of 0.5 to 3% by weight. In embodiments, the permeation enhancer comprises both permeation enhancing and antioxidant properties. For example, vitamin E may include both permeation enhancing and antioxidant characteristics. In embodiments, the permeation enhancer comprises one of or more of bile salt, cetylpyridinium chloride (CPC), sodium lauryl sulfate (SLS), Tween 80, L-menthol, dimethyl sulfoxide (DMSO), oleic alcohol, oleic acid, oleyl oleate, levulinic acid, propylene glycol, dipropylene glycol, ethanol, or any combination thereof. In embodiments, the composition comprises other stability enhancers including antioxidants or chelators. In embodiments, the stability enhancer comprises one or more of a- tocopherol, tocopherol acetate, L-Glutathione, L-cysteine, ascorbic acid, ascorbyl palmitate, propyl gallate, butylated hydroxytoluene (BHT), butylated hydroxyanisole (BHA), Tocobiol, Ethylenediaminetetraacetic acid (EDTA), or any combination thereof.

In embodiments, the mucoadhesive composition comprises a buffering agent, and/or a saliva stimulating agent. In embodiments, the buffering agent comprises one or more of citric acid, tartaric acid, fumaric acid, succinic acid, malic acid, sodium citrate, sodium tartrate, sodium succinate, sodium fumarate, or any combination thereof. In embodiments, the buffering agent is used with a pH adjusting agent, such as sodium hydroxide. In embodiments, the saliva stimulating agent comprises one or more of citric acid, malic acid, lactic acid, ascorbic acid, tartaric acid, or any combination thereof.

In embodiments, the mucoadhesive composition comprises a sweetener and/or flavoring agent. In embodiments, the sweetening agent comprises one or more of Advantame, sucrose, dextrose, fructose, glucose, maltose, maltitol, saccharin, sucralose, neotame, cyclamate, aspartame, acesulfame-K, or any combination thereof. In embodiments, the flavoring agent comprises one or more of lime flavor, lemon flavor, peppermint oil, cinnamon oil, vanilla extract, menthol, L-menthol, or any combination thereof.

In embodiments, the mucoadhesive composition comprises a coloring agent. In embodiments, the coloring agent comprises one or more of D&C or FD&C red, yellow, green, blue, iron oxide red, iron oxide yellow, titanium dioxide, or any combination thereof.

In embodiments, the mucoadhesive composition comprises a polymeric carrier matrix that is an oral transmucosal film. In embodiments, the oral transmucosal film has a thickness in the range of 0.01 mm to 1.5 mm, and more preferably within the range of 0.5 mm to 1.5 mm.

In embodiments, the oral transmucosal film of the present disclosure comprises an active layer and a backing layer. In embodiments, the composition of the active layer film is shown in Table 1.

TABLE 1
Composition of the active layer film

| wt % ranges | Components |
|---|---|
| 32-40 wt % | DMT (N,N-dimethyltryptamine) |
| 33-40 wt % | polymeric carrier matrix |
| 5-15 wt % | D-Maltitol |
| 2-10 wt % | citric acid |
| 1-5 wt % | TPGS (vitamin E) |
| 0.5-3 wt % | L-Glutathione |

In embodiments, the composition of the active layer film is shown in Table 2.

TABLE 2
Composition of the active layer film

| wt % ranges | Components |
|---|---|
| 35.85 wt % | DMT (N,N-dimethyltryptamine) |
| 23.10 wt % | Nisso HPC SSL |
| 11.15 wt % | D-Maltitol |
| 5.69 wt % | Nisso HPC-L |
| 5.03 wt % | citric acid |
| 3.58 wt % | TPGS (vitamin E) |
| 3.41 wt % | CMC (ashland 7LF) |
| 3.11 wt % | Arome Type Suppresseur amertume |
| 2.61 wt % | Plasdone S-630 |
| 2.11 wt % | L-Glutathione |
| 1.59 wt % | Evospray natural Lime flavor |
| 1.50 wt % | Sucralose |
| 1.14 wt % | Klucel HPC-GXF (homo 5 min 10K rpm) |
| 0.12 wt % | Advantame |
| 0.01 wt % | FD&C Yellow No. 06 powder |

An exemplary composition of the 40 mg DMT buccal film (i.e., oral thin film) is shown in Table 3.

TABLE 3
40 mg DMT buccal film composition

| | | Formulation | Quantity (40 mg |
|---|---|---|---|
| Compounds | Grade | composition (%) | DMT film) |
| N,N-dimethyltryptamine | — | 27.15 | 40.00 |
| Sucralose | USP | 0.56 | 0.83 |
| L-Glutathione | EP | 1.29 | 1.90 |
| L-Menthol | USP | 1.30 | 1.92 |
| PEG 300 | NF, EP | 1.72 | 2.53 |
| Citric Acid Anhydrous | USP | 8.09 | 11.92 |
| Maltitol | NF | 5.43 | 8.00 |
| Hydroxypropyl Cellulose | USP, JP, EP | 31.46 | 46.35 |
| Copovidone | USP, JP, EP | 15.75 | 23.20 |
| Hydroxypropyl Methylcellulose | USP, JP, EP | 7.24 | 10.67 |
| (HPMC E50) | | | |
| Methanol | NF | — | Q.S. |
| Purified Water | USP | — | Q.S. |
| Total | N/A | 100 | 147.32 |

Abbreviations: DMT: N,N-dimethyltryptamine; EP = European Pharmacopoeia; JP = Japanese Pharmacopoeia; N/A = Not applicable; NF = National Formulary; Q.S. = Quantity Sufficient; USP = United States Pharmacopoeia.

An exemplary composition of the 80 mg DMT buccal film is shown in Table 4.

TABLE 4
80 mg DMT buccal film composition

| | | Formulation | Quantity (80 mg |
|---|---|---|---|
| Compounds | Grade | composition ( %) | DMT film) |
| N,N-dimethyltryptamine | — | 35.846 | 80.00 |
| Sucralose | USP | 1.502 | 3.35 |
| Adventame | FCC/USP | 0.119 | 0.27 |
| L-Glutathione | USP | 2.105 | 4.70 |
| Vitamin E TPGS, NF | USP/NF | 3.585 | 8.00 |
| Evospray Natural Lime Flavor | USP | 1.593 | 3.56 |
| Arome Type Suppressor | In house, USP | 3.107 | 6.93 |
| Amertume | | | |
| FD&C Yellow No. 06 Powder | USP | 0.012 | 0.03 |
| Citric Acid Anhydrous | USP | 5.030 | 11.23 |
| Maltitol (SweetPearl P90) | FCC/USP | 11.152 | 24.89 |
| Copovidone (Plasdone | USP | 2.606 | 5.82 |
| S-630) | | | |
| Hydroxypropyl Cellulose | USP | 23.101 | 51.56 |
| (Nisso HPC-SSL) | | | |
| Hydroxypropyl Cellulose | USP | 5.690 | 12.70 |
| (Nisso HPC-L) | | | |
| Hydroxypropyl Cellulose | USP | 1.138 | 2.54 |
| (Klucel GXF) | | | |
| Sodium | USP | 3.414 | 7.62 |
| Carboxymethylcellulose | | | |
| (Aqualon CMC 7LF PH) | | | |
| Methyl Alcohol | USP/Ph, Eur. | — | Q.S. |
| Purified Water | USP | — | Q.S. |
| Total | N/A | 100 | 223.20 |

Abbreviations: DMT: N,N-dimethyltryptamine; FCC = Food Chemicals Codex; FD&C = Food, Drug and Cosmetics; N/A = Not applicable; NF = National Formulary; Ph. Eur. = The European Pharmacopoeia; Q.S. = Quantity Sufficient; TPGS = D-α-Tocopherol Polyethylene glycol 1000 Succinate; USP = United States Pharmacopoeia.

In embodiments, the mucoadhesive composition comprises a backing layer and a DMT buccal film composition as described herein. In embodiments, the backing layer is adapted to reduce or prevent DMT from being transported across the backing layer while the mucoadhesive therapeutic product is being administered. In embodiments, the backing layer comprises a polymeric carrier matrix and a pigment. In embodiments, the backing layer comprises carboxymethyl cellulose (CMC), hydroxypropyl cellulose (HPC), and a copolymer of N-vinyl-2-pyrrolidone and vinyl acetate.

In embodiments, the backing layer film composition is shown in Table 5.

TABLE 5
Composition of the backing layer film

| wt % ranges | Components |
|---|---|
| 75-85 wt % | mucoadhesive polymeric matrix |
| 10-20 wt % | PEG |
| 1-3 wt % | Titanium dioxide (TiO₂) |
| 0-7 wt % | other |

In embodiments, the backing layer film composition is shown in Table 6.

TABLE 6
Composition of the backing layer film

| wt % ranges | Components |
|---|---|
| 68.42 wt % | Nisso HPC-L |
| 7.80 wt % | CMC (ashland 7LF) |
| 5.96 wt % | Plasdone S-630 |
| 14.31 wt % | PEG300 |
| 2.08 wt % | Titanium dioxide (TiO₂) |
| 1.43 wt % | NaOH 50% |

An exemplary composition of the backing layer film for the 80 mg DMT dual layer buccal film is shown in Table 7.

TABLE 7
Backing layer film composition for 80 mg DMT buccal film

| | | Formulation | Quantity (80 mg |
|---|---|---|---|
| Compounds | Grade | composition (%) | DMT film) |
| Sodium Hydroxide NF | USP | 0.72 | 0.38 |
| Titanium Oxide | USP | 2.10 | 1.09 |
| Copovidone | USP | 6.00 | 3.14 |
| (Plasdone S-630) | | | |
| Hydroxypropyl Cellulose | USP | 68.91 | 36.01 |
| (Nisso HPC-L) | | | |
| Sodium | USP | 7.86 | 4.11 |
| Carboxymethylcellulose | | | |
| (Aqualon CMC 7LF PH) | | | |
| PEG 300 | USP/Ph, Eur. | 14.41 | 7.53 |
| Purified Water | USP | — | Q.S. |
| Total | N/A | 100 | 52.25 |

Abbreviations: DMT: N,N-dimethyltryptamine; N/A = Not applicable; PEG 300 = Polyethylene glycol 300; Ph. Eur. = The European Pharmacopoeia; Q.S. = Quantity Sufficient; USP = United States Pharmacopoeia.

In embodiments, the composition of the present disclosure comprises about 5 mg to about 400 mg of DMT or a pharmaceutically acceptable salt thereof, for example, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg of DMT or a pharmaceutically acceptable salt thereof.

In embodiments, the composition of the present disclosure comprises about 5 mg to about 400 mg of DMT or a pharmaceutically acceptable salt thereof. In embodiments, the composition comprises about 20 mg to about 200 mg of DMT or a pharmaceutically acceptable salt thereof. In embodiments, the composition comprises about 60 mg to about 200 mg of DMT or a pharmaceutically acceptable salt thereof. In embodiments, the composition comprises about 80 mg to about 200 mg of DMT or a pharmaceutically acceptable salt thereof. In embodiments, the composition comprises about 120 mg to about 200 mg of DMT or a pharmaceutically acceptable salt thereof. In embodiments, the composition comprises about 160 mg to about 200 mg of DMT or a pharmaceutically acceptable salt thereof.

### Methods of Treatment

In embodiments, about 5 mg to about 400 mg of DMT free base or an equivalent dose of the pharmaceutically acceptable salt thereof is administered to the patient. In embodiments, the therapeutically effective amount comprises a dose ranging from about 5 mg to about 400 mg DMT free base or an equivalent dose of the pharmaceutically acceptable salt thereof. In embodiments, the therapeutically effective amount comprises a dose ranging from about 5 mg to about 400 mg, or about 10 mg to about 400 mg, or about 20 mg to about 200 mg, or about 40 mg to about 160 mg, or about 80 mg to about 120 mg DMT free base or an equivalent dose of the pharmaceutically acceptable salt thereof. In embodiments, the therapeutically effective amount comprises a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, or more DMT free base or an equivalent dose of the pharmaceutically acceptable salt thereof.

The effective amounts may be provided as a single dose or on regular schedule, i.e., on a daily, weekly, monthly, or yearly basis or an irregular schedule with varying administration days, weeks, months, etc. In embodiments, a patient is administered the composition of the present disclosure multiple times over a set a period of time. In embodiments, the patient is administered the composition once every time period of between about 2 weeks and about 12 months for a set treatment period. In embodiments, the patient is administered the composition once every time period of between about 2 weeks and about 6 months for a set treatment period. In embodiments, the patient is administered the composition once every about 2 weeks, about 3 weeks, about 4 weeks, about 2 months, about 3 months, about 4 months, about 5 months, about 6 months, about 7 months, about 8 months, about 9 months, about 10 months, about 11 months, or about 12 months for a set treatment period. In embodiments, the patient is administered the composition once every about 2 weeks for a set treatment period. In embodiments, the patient is administered the composition once every month for a set treatment period. In embodiments, the patient is administered the composition once every about 6 weeks for a set treatment period. In embodiments, the patient is administered the composition once every about 2 months for a set treatment period. In embodiments, the patient is administered the composition once every about 3 months for a set treatment period. In embodiments, the patient is administered the composition once every about 6 months for a set treatment period.

To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In aspects, the therapeutically effective amount for the first dose is higher than the therapeutically effective amount for one or more of the subsequent doses. In aspects, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In embodiments, the administration of the composition of the present disclosure is buccal, sublingual, gingival, on the tongue, nasal, vaginal, or a rectal administration. In embodiments, the administration of the composition of the present disclosure is buccal or sublingual.

In embodiments, the administration (e.g., buccal administration) of the compositions of the present disclosure provide a Tₘₐₓ of DMT of about 0.1 h to about 4 h (such as about 0.3 h to about 2 h), for example, about 0.1 h, about 0.2 h, about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition.

In embodiments, the administration (e.g., buccal administration) of the compositions of the present disclosure provide a Tₘₐₓ of DMT of about 10 min to about 240 min, for example, about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the administration (e.g., buccal administration) of the compositions of the present disclosure provide a T_{1/2} of DMT of about 0.1 h to about 2 h (such as about 0.3 h to about 1h), for example, about 0.1 h, about 0.2 h, about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, or about 2.0 h, including any values or ranges therebetween following administration of the composition.

In embodiments, the administration (e.g., buccal administration) of the compositions of the present disclosure provide a Tₘₐₓ of N,N-dimethyltryptamine-N-oxide (DMT-NO) of about 1 h to about 4 h, for example, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween, following administration of the composition.

In embodiments, the administration (e.g., buccal administration) of the compositions of the present disclosure provide a Tₘₐₓ of DMT-NO of about 60 min to about 240 min, for example, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the administration (e.g., buccal administration) of the compositions of the present disclosure provide a T_{1/2} of DMT-NO of about 1 h to about 8 h, for example, about 1 h, about 2 h, about 3 h, about 4 h, about 5 h, about 6 h, about 7 h, or about 8 h, including any values or ranges therebetween following administration of the composition.

In embodiments, the administration (e.g., buccal administration) of the compositions of the present disclosure provide a Tₘₐₓ of indole-3-acetic acid (IAA) of about 1 h to about 4 h, for example, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition.

In embodiments, the administration (e.g., buccal administration) of the compositions of the present disclosure provide a Tₘₐₓ of IAA of about 60 min to about 240 min, for example, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the administration (e.g., buccal administration) of the compositions of the present disclosure provide a T_{1/2} of IAA of about 1 h to about 8 h, for example, about 1 h, about 2 h, about 3 h, about 4 h, about 5 h, about 6 h, about 7 h, or about 8 h, including any values or ranges therebetween following administration of the composition.

In embodiments, the administration (e.g., buccal administration) of the composition provides a Cₘₐₓ of DMT of about 1 ng/mL to about 200 ng/mL, for example, about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/ml, about 50 ng/ml, about 55 ng/ml, about 60 ng/mL, about 65 ng/mL, about 70 ng/mL, about 75 ng/mL, about 80 ng/ml, about 85 ng/mL, about 90 ng/mL, about 95 ng/mL, about 100 ng/mL, about 105 ng/ml, about 110 ng/mL, about 115 ng/mL, about 120 ng/mL, about 125 ng/mL, about 130 ng/mL, about 135 ng/mL, about 140 ng/mL, about 145 ng/ml, about 150 ng/mL, about 155 ng/mL, about 160 ng/mL, about 165 ng/ml, about 170 ng/mL, about 175 ng/mL, about 180 ng/ml, about 185 ng/mL, about 190 ng/mL, about 195 ng/mL, or about 200 ng/mL, including any values or ranges therebetween, following administration of the composition.

In embodiments, the administration (e.g., buccal administration) of the composition provides a Cₘₐₓ of DMT-NO of about 1 ng/mL to about 200 ng/ml, for example, about 1 ng/mL, about 5 ng/mL, about 10 ng/ml, about 15 ng/mL, about 20 ng/ml, about 25 ng/mL, about 30 ng/mL, about 35 ng/mL, about 40 ng/ml, about 45 ng/mL, about 50 ng/mL, about 55 ng/mL, about 60 ng/mL, about 65 ng/mL, about 70 ng/mL, about 75 ng/ml, about 80 ng/ml, about 85 ng/ml, about 90 ng/ml, about 95 ng/mL, about 100 ng/mL, about 105 ng/ml, about 110 ng/mL, about 115 ng/ml, about 120 ng/mL, about 125 ng/mL, about 130 ng/ml, about 135 ng/mL, about 140 ng/mL, about 145 ng/mL, about 150 ng/mL, about 155 ng/ml, about 160 ng/mL, about 165 ng/ml, about 170 ng/mL, about 175 ng/ml, about 180 ng/mL, about 185 ng/mL, about 190 ng/mL, about 195 ng/mL, or about 200 ng/mL, including any values or ranges therebetween, following administration of the composition.

In embodiments, the administration (e.g., buccal administration) of the composition provides a Cₘₐₓ of IAA of about 1,000 ng/mL to about 20,000 ng/mL, for example, about 1,000 ng/mL, about 1,500 ng/mL, about 2,000 ng/mL, about 2,500 ng/mL, about 3,000 ng/mL, about 3,500 ng/mL, about 4,000 ng/mL, about 4,500 ng/mL, about 5,000 ng/mL, about 5,500 ng/ml, about 6,000 ng/mL, about 6,500 ng/mL, about 7,000 ng/mL, about 7,500 ng/mL, about 8,000 ng/mL, about 8,500 ng/mL, about 9,000 ng/mL, about 9,500 ng/mL, about 10,000 ng/ml, about 10,500 ng/mL, about 11,000 ng/mL, about 11,500 ng/mL, about 12,000 ng/ml, about 12,500 ng/mL, about 13,000 ng/mL, about 13,500 ng/mL, about 14,000 ng/mL, about 14,500 ng/mL, about 15,000 ng/mL, about 15,500 ng/mL, about 16,000 ng/mL, about 16,500 ng/ml, about 17,000 ng/mL, about 17,500 ng/mL, about 18,000 ng/mL, about 18,500 ng/mL, about 19,000 ng/mL, about 19,500 ng/mL, or about 20,000 ng/mL, including any values or ranges therebetween, following administration of the composition.

In embodiments, the administration (e.g., buccal administration) of the composition provides a AUCₗₐₛₜ of DMT of about 1 ng·h/mL to about 100 ng·h/mL, for example, about 1 ng·h/mL, about 2 ng·h/mL, about 3 ng·h/mL, about 4 ng·h/mL, about 5 ng·h/mL, about 10 ng·h/mL, about 15 ng·h/mL, about 20 ng·h/mL, about 25 ng·h/mL, about 30 ng·h/mL, about 35 ng·h/mL, about 40 ng·h/mL, about 45 ng·h/mL, about 50 ng·h/mL, about 55 ng·h/mL, 60 ng·h/mL, about 65 ng·h/mL, about 70 ng·h/mL, about 75 ng·h/mL, about 80 ng·h/mL, about 85 ng·h/mL, about 90 ng·h/mL, about 95 ng·h/mL, or about 100 ng·h/mL, including any values or ranges therebetween, following administration of the composition.

In embodiments, the administration (e.g., buccal administration) of the composition provides a AUCₗₐₛₜ of DMT-NO of about 1 ng·h/mL to about 100 ng·h/mL, for example, about 1 ng·h/mL, about 2 ng·h/mL, about 3 ng·h/mL, about 4 ng·h/mL, about 5 ng·h/mL, about 10 ng·h/mL, about 15 ng·h/mL, about 20 ng·h/mL, about 25 ng·h/mL, about 30 ng·h/mL, about 35 ng·h/mL, about 40 ng·h/mL, about 45 ng·h/mL, about 50 ng·h/mL, about 55 ng·h/mL, 60 ng·h/mL, about 65 ng·h/mL, about 70 ng·h/mL, about 75 ng·h/mL, about 80 ng·h/mL, about 85 ng·h/mL, about 90 ng·h/mL, about 95 ng·h/mL, or about 100 ng·h/mL, including any values or ranges therebetween, following administration of the composition.

In embodiments, the administration (e.g., buccal administration) of the composition provides a AUCₗₐₛₜ of IAA of about 5,000 ng·h/mL to about 50,000 ng·h/mL, for example, about 5,000 ng·h/mL, about 6,000 ng·h/mL, about 7,000 ng·h/mL, about 8,000 ng·h/mL, about 9,000 ng·h/mL, about 10,000 ng·h/mL, about 11,000 ng·h/mL, about 12,000 ng·h/mL, about 13,000 ng·h/mL, about 14,000 ng·h/mL, about 15,000 ng·h/mL, about 16,000 ng·h/mL, about 17,000 ng·h/mL, about 18,000 ng·h/mL, about 19,000 ng·h/mL, about 20,000 ng·h/mL, about 21,000 ng·h/mL, about 22,000 ng·h/mL, about 23,000 ng·h/mL, about 24,000 ng·h/mL, about 25,000 ng·h/mL, about 26,000 ng·h/mL, about 27,000 ng·h/mL, about 28,000 ng·h/mL, about 29,000 ng·h/mL, about 30,000 ng·h/mL, about 31,000 ng·h/mL, about 32,000 ng·h/mL, about 33,000 ng·h/mL, about 34,000 ng·h/mL, about 35,000 ng·h/mL, about 36,000 ng·h/mL, about 37,000 ng·h/mL, about 38,000 ng·h/mL, about 39,000 ng·h/mL, about 40,000 ng·h/mL, about 41,000 ng·h/mL, about 42,000 ng·h/mL, about 43,000 ng·h/mL, about 44,000 ng·h/mL, about 45,000 ng·h/mL, about 46,000 ng·h/mL, about 47,000 ng·h/mL, about 48,000 ng·h/mL, about 49,000 ng·h/mL, about or 50,000 ng·h/mL, including any values or ranges therebetween, following administration of the composition.

In embodiments, the compositions of the present disclosure comprise about 20 mg to about 200 mg of DMT or a pharmaceutically acceptable salt thereof, wherein the administration provides a DMT Tₘₐₓ of about 0.3 h to about 2 h and a DMT Cmax of about 5 ng/mL to about 80 ng/mL following administration. In embodiments, the DMT Tmax is about about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition. In embodiments, the DMT Cmax is about 5 ng/ml, about 10 ng/ml, about 15 ng/mL, about 20 ng/mL, about 25 ng/mL, about 30 ng/mL, about 35 ng/ml, about 40 ng/ml, about 45 ng/mL, about 50 ng/mL, about 55 ng/mL, about 60 ng/mL, about 65 ng/ml, about 70 ng/mL, about 75 ng/mL, or about 80 ng/mL, including any values or ranges therebetween, following administration of the composition.

In embodiments, the compositions of the present disclosure comprise about 20 mg to about 200 mg of DMT or a pharmaceutically acceptable salt thereof, wherein the administration provides a DMT Tₘₐₓ of about 0.3 h to about 2 h and a DMT AUClast of about 3 ng·h/mL to about 70 ng·h/mL following administration. In embodiments, the DMT Tmax is about about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition. In embodiments, the DMT AUClast is about 3 ng·h/mL, about 4 ng·h/mL, about 5 ng·h/mL, about 10 ng·h/mL, about 15 ng·h/mL, about 20 ng·h/mL, about 25 ng·h/mL, about 30 ng·h/mL, about 35 ng·h/mL, about 40 ng·h/mL, about 45 ng·h/mL, about 50 ng·h/mL, about 55 ng·h/mL, 60 ng·h/mL, about 65 ng·h/mL, about 70 ng·h/mL, about 75 ng·h/mL, about 80 ng·h/mL, about 85 ng·h/mL, about 90 ng·h/mL, about 95 ng·h/mL, or about 100 ng·h/mL, including any values or ranges therebetween, following administration of the composition.

In embodiments, the present disclosure provides a two-phase dosing regimen for the treatment of TRD using DMT or a pharmaceutically acceptable salt thereof. In embodiments, the regimen comprises two phases: an induction phase and a maintenance phase. The induction phase induces remission of the patient's TRD through the administration of one or more DMT induction doses. In embodiments, the induction phase continues until the patient achieves remission of TRD symptoms as determined by one or more psychiatric evaluation tools (e.g., assessed by patient's MADRS total score equal to or less than 10). Following induction, the patient transitions to the maintenance phase comprising the administration of DMT maintenance doses. The maintenance phase sustains remission, reduces the incidence of relapse, and/or supports long-term therapeutic benefit. The dosing parameters in the maintenance phase—such as dose amount and frequency—may be the same as or different relative to those used during the induction phase.

In embodiments, the present disclosure provides methods of treating TRD by administering DMT or a pharmaceutically acceptable salt thereof to a patient in need thereof, the method comprising: a) administering to the patient an induction regimen comprising a DMT induction dose; and b) thereafter administering to the patient a maintenance regimen comprising a DMT maintenance dose.

In embodiments, the induction regimen comprises one DMT dose. In embodiments, the induction regimen comprises multiple DMT doses.

In embodiments, the DMT induction doses are administered at an interval of between about 1 week and about 12 weeks between the doses. In embodiments, the DMT induction doses are administered between once a week and once every 12 weeks. In embodiments, the induction doses are administered once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks.

In embodiments, the induction regimen is about 1 week to about 24 weeks. In embodiments, the induction regimen is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, or about 24 weeks.

In embodiments, the induction regimen comprises about 1 to about 10 DMT induction doses. In embodiments, the induction regimen comprises about 1, about 2, about 3, about 4, about 5, about 6, about 7, about 8, about 9, or about 10 once a month DMT induction doses.

In embodiments, the induction regimen comprises about 1 to about 10 DMT induction doses once every 2 weeks. In embodiments, the induction regimen comprises about 1, about 2, about 3, about 4, about 5, about 6, about 7, about 8, about 9, or about 10 DMT induction doses once every 2 weeks.

In embodiments, the DMT induction dose is from about 5 mg to about 400 mg, about 20 mg to about 200 mg, about 40 mg to about 160 mg, or about 80 mg to about 120 mg of DMT free base or an equivalent amount of the pharmaceutically acceptable salt thereof. In embodiments, the DMT induction dose is about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of DMT free base or an equivalent amount of the pharmaceutically acceptable salt thereof.

In embodiments, the DMT induction regimen comprises multiple induction doses, each independently comprising from about 5 mg to about 400 mg, about 20 mg to about 200 mg, about 40 mg to about 160 mg, or about 80 mg to about 120 mg of DMT free base or an equivalent amount of the pharmaceutically acceptable salt thereof. In embodiments, the DMT induction regimen comprises multiple induction doses, each independently selected from about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, and about 200 mg of DMT free base or an equivalent amount of the pharmaceutically acceptable salt thereof. In embodiments, the DMT induction regimen comprises multiple induction doses, each comprising the same amount of DMT free base or an equivalent amount of the pharmaceutically acceptable salt thereof. In embodiments, the DMT induction regimen comprises multiple induction doses, each comprising a different amount of DMT free base or an equivalent amount of the pharmaceutically acceptable salt thereof. In embodiments, the DMT induction regimen comprises multiple induction doses, each comprising the same, a different amount of DMT free base or an equivalent amount of a pharmaceutically acceptable salt thereof, or a combination thereof. In embodiments, the DMT induction regimen comprises two induction doses each comprising about 120 mg of DMT free base or an equivalent amount of the pharmaceutically acceptable salt thereof.

In embodiments, the induction regimen provides remission of the patient's TRD. In embodiments, the induction regimen provides a reduction in the patient's MADRS total score by at least about 20%, at least about 30%, at least about 40%, or at least about 50% compared to prior to the administration. In embodiments, the induction regimen provides a reduction in the patient's MADRS total score by at least about 50% compared to prior to the administration. In embodiments, the induction regimen provides a reduction in the patient's MADRS total score by at least about 6 compared to prior to the administration.

In embodiments, the maintenance regimen comprises one DMT maintenance dose. In embodiments, the maintenance regimen comprises multiple DMT maintenance doses.

In embodiments, the DMT maintenance doses are administered at an interval of between about 1 week and about 12 weeks between the doses. In embodiments, the DMT maintenance doses are administered between once a week and once every 12 weeks. In embodiments, the DMT maintenance doses are administered between once a week and once every 12 weeks. In embodiments, the induction doses are administered once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In embodiments, the DMT maintenance doses are administered once a month, once every 2 months, once every 3 months, or once every 4 months.

In embodiments, the maintenance regimen is about 1 week to about 96 weeks. In embodiments, the maintenance regimen is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 53 week, about 54 weeks, about 55 weeks, about 56 weeks, about 57 weeks, about 58 weeks, about 59 weeks, about 60 weeks, about 61 weeks, about 62 weeks, about 63 weeks, about 64 weeks, about 65 weeks, about 66 weeks, about 67 weeks, about 68 weeks, about 69 weeks, about 70 weeks, about 71 weeks, about 72 weeks, about 73 weeks, about 74 weeks, about 75 weeks, about 76 weeks, about 77 weeks, about 78 weeks, about 79 weeks, about 80 weeks, about 81 weeks, about 82 weeks, about 83 weeks, about 84 weeks, about 85 weeks, about 86 weeks, about 87 weeks, about 88 weeks, about 89 weeks, about 90 weeks, about 91 weeks, about 92 weeks, about 93 weeks, about 94 weeks, about 95 weeks, about 96 weeks, about 97 weeks, about 98 weeks, or more weeks. In embodiments, the maintenance regimen is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, or more months.

In embodiments, the maintenance regimen comprises about 1 to about 20 DMT maintenance doses. In embodiments, the maintenance regimen comprises about 1, about 2, about 3, about 4, about 5, about 6, about 7, about 8, about 9, about 10, about 11, about 12, about 13, about 14, about 15, about 16, about 17, about 18, about 19, about 20, or more maintenance doses.

In embodiments, the maintenance regimen comprises 1 to about 20 once a week DMT maintenance doses. In embodiments, the maintenance regimen comprises about 1, about 2, about 3, about 4, about 5, about 6, about 7, about 8, about 9, about 10, about 11, about 12, about 13, about 14, about 15, about 16, about 17, about 18, about 19, about 20, or more once a week DMT maintenance doses. In embodiments, the maintenance regimen comprises about 1 to about 10 once a month DMT maintenance doses. In embodiments, the maintenance regimen comprises about 1, about 2, about 3, about 4, about 5, about 6, about 7, about 8, about 9, about 10, about 11, about 12, about 13, about 14, about 15, about 16, about 17, about 18, about 19, about 20, or more once a month DMT maintenance doses.

In embodiments, the DMT maintenance regimen is administered chronically to maintain remission of the patient's TRD.

In embodiments, the DMT maintenance dose is from about 5 mg to about 400 mg, about 10 mg to about 200 mg, or about 40 mg to about 160 mg, or about 80 mg to about 120 mg of DMT free base or an equivalent amount of the pharmaceutically acceptable salt thereof. In embodiments, the DMT maintenance dose is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of DMT free base or an equivalent amount of the pharmaceutically acceptable salt thereof. In embodiments, the DMT maintenance dose is about 60 mg of DMT free base or an equivalent amount of the pharmaceutically acceptable salt thereof. In embodiments, the DMT maintenance dose is about 120 mg of DMT free base or an equivalent amount of the pharmaceutically acceptable salt thereof.

In embodiments, the DMT maintenance dose is about 10%, about 20%, about 30%, about 40%, about 50%, about 60%, about 70%, about 80%, about 90%, or about 100% of the DMT induction dose.

In embodiments, the first DMT maintenance dose is administered about 1 week to about 24 weeks after the last DMT induction dose. In embodiments, the first DMT maintenance dose is administered about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after the last DMT induction dose.

In embodiments, the maintenance regimen maintains remission of the patient's TRD.

In embodiments, the present disclosure provides methods of treating treatment resistant depression in a patient in need thereof, wherein the method comprises: a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and b) thereafter administering to the patient a DMT maintenance dose of about 120 mg of DMT free base or an equivalent amount of the pharmaceutically acceptable salt thereof.

In embodiments, the present disclosure provides methods of treating treatment resistant depression in a patient in need thereof, wherein the method comprises: a) administering to the patient 2 DMT induction doses each comprising about 120 mg of DMT free base or an equivalent amount of the pharmaceutically acceptable salt thereof, wherein the DMT induction doses are administered once every 2 weeks; and b) thereafter administering to the patient a DMT maintenance dose of about 120 mg of DMT free base or an equivalent amount of the pharmaceutically acceptable salt thereof. In embodiments, the maintenance dose is administered about 12 weeks after the last induction dose.

In embodiments, the present disclosure provides methods of treating treatment resistant depression in a patient in need thereof, wherein the method comprises: a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and) thereafter administering to the patient a DMT maintenance dose of about 60 mg of DMT free base or an equivalent amount of the pharmaceutically acceptable salt thereof.

In embodiments, the present disclosure provides methods of treating treatment resistant depression in a patient in need thereof, wherein the method comprises: a) administering to the patient 2 DMT induction doses each comprising about 120 mg of DMT free base or an equivalent amount of the pharmaceutically acceptable salt thereof, wherein the DMT induction doses are administered once every 2 weeks; and b) thereafter administering to the patient a DMT maintenance dose of about 60 mg of DMT free base or an equivalent amount of the pharmaceutically acceptable salt thereof. In embodiments, the maintenance dose is administered about 12 weeks after the last induction dose.

In embodiments, the present disclosure provides methods of treating treatment resistant depression in a patient in need thereof, wherein the method comprises: a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and b) thereafter administering to the patient about 1 to about 20 once a week DMT maintenance doses each comprising about 60 mg to about 120 mg of DMT free base or an equivalent amount of the pharmaceutically acceptable salt thereof.

In embodiments, the present disclosure provides methods of treating treatment resistant depression in a patient in need thereof, wherein the method comprises: a) administering to the patient 1 to about 10 DMT induction doses each comprising about 120 mg of DMT free base or an equivalent amount of the pharmaceutically acceptable salt thereof, wherein the DMT induction doses are administered between once a week to once every 3 weeks; and b) thereafter administering to the patient 1 to about 20 once a month DMT maintenance doses each comprising about 60 mg to about 120 mg of DMT free base or an equivalent amount of the pharmaceutically acceptable salt thereof.

In embodiments, the present disclosure provides methods of treating treatment resistant depression in a patient in need thereof, wherein the method comprises: a) administering to the patient a DMT induction dose once every 4 weeks; and b) thereafter administering to the patient a DMT maintenance dose once a month.

In embodiments, the present disclosure provides methods of treating treatment resistant depression in a patient in need thereof, wherein the method comprises: a) administering to the patient 2 DMT induction doses once every 4 weeks; and b) thereafter administering to the patient 5 DMT maintenance doses once a month.

In embodiments, the present disclosure provides methods of treating TRD in a patient in need thereof (e.g., a patient whose TRD symptoms recur during the maintenance regimen), wherein the method comprises administereing a second induction regimen following completion of a maintenance regimen. In embodiments, the second induction regimen is initiated in response to a relapse or recurrence of symptoms, or upon determination that the maintenance regimen was insufficient to sustain remission. In embodiments, the second induction regimen comprises one or more DMT induction doses, administered in accordance with any of the induction regimens described herein. In embodiments, the second induction regimen is the same or substantially similar to the initial induction regimen. In embodiments, the dosing, frequency, or duration of the second induction regimen is adjusted based on the patient's response to prior treatment. In embodiments, the second induction regimen is followed by a second maintenance regimen. In embodiments, second maintenance regimen is the same or substantially similar to the first maintenance regimen. In embodiments, the dosing, frequency, or duration of the second maintenance regimen is adjusted based on the patient's response to prior treatment.

In embodiments, the present disclosure provide individualized treatment regimens for the treatment of TRD, tailored to the specific therapeutic needs and clinical responses of individual patients. In embodiments, the patient's regimen optimizes the efficacy and safety of DMT or a pharmaceutically acceptable salt thereof, by adjusting the patient's DMT dose, dosing frequency, treatment duration, and time between induction and maintenance phases based on patient-specific factors (e.g., age, sex, weight, and depression score).

In embodiments, the individualized treatment regimen comprises administering DMT induction doses at a frequency determined by the patient's clinical profile, symptom severity, and response to treatment. In embodiments, the induction phase continues until the patient achieves clinically remission of TRD symptoms, as assessed by psychiatric evaluation tools in the art (e.g., assessed by patient's MADRS total score equal to or less than 10). Following the induction phase, and upon achieving remission, the patient transitions to a maintenance regimen. The maintenance regimen is intended to sustain remission, prevent relapse, and maintain long-term therapeutic benefit. In embodiments, the maintenance regimen comprises administering DMT maintenance doses at the same or a lower dose amount and/or at the same or a reduced frequency relative to the induction phase.

In embodiments, the present disclosure provide methods of treating TRD by administering DMT or a pharmaceutically acceptable salt thereof to a patient in need thereof, the method comprising: a) administering to the patient an induction regimen comprising a DMT induction dose, wherein the induction regimen provides remission of the patient's TRD; and b) thereafter administering to the patient a maintenance regimen comprising a DMT maintenance dose.

### Methods of Use

In aspects, the present disclosure provides methods of treatment of a disease or disorder, wherein the method comprises administering a therapeutically effective amount of the composition of the present disclosure to a patient in need thereof. The disease or disorder is neurological disease or condition, for example, treatment-resistant depression (TRD).

In embodiments, the present disclosure provides methods of treating other neurological diseases or conditions. In embodiments, the neurological disease or condition is selected from: a neuropsychiatric disorder, such as depressive disorder or depression (including severe depression such as treatment-resistant depression (TRD), major depressive disorder (MDD) and persistent depressive disorder), alexithymia, catatonic depression, a depressive disorder due to a medical condition, postpartum depression, premenstrual dysphoric disorder, seasonal affective disorder, anxiety, anxiety disorder, social anxiety disorder (SAD), general anxiety disorder (GAD), avolition disorder, bipolar disorder (including bipolar I disorder and bipolar II disorder), post-traumatic stress disorder (PTSD), body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, and schizotypal schizoid disorders, suicide ideation, rumination/unproductive repetitive thoughts negatively impacting one's behavior, mood, and/or ability to focus, obsessive-compulsive disorder, addiction (including substance use disorders, such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography, videogames, work, exercise, spiritual obsession, self-harm, travel and shopping addiction, etc.), eating disorders (including anorexia nervosa, bulimia nervosa and binge eating disorder), pain (including pain associated with migraine or headache or chronic pain), prolonged grief disorder, paranoid personality disorder, Autoimmune OCD (adult), behavioral symptoms/anxiety in Fragile X (adult), or Xenomelia.

In embodiments, the present disclosure provide methods of treating treatment-resistant depression (TRD) in a patient in need thereof. In embodiments, a patient with treatment-resistant depression has not responded adequately to other TRD therapies. In embodiments, patients with treatment-resistant depression have been diagnosed with major depressive disorder (MDD) and have been unable to respond to three or more treatment attempts. In embodiments, patients with treatment-resistant depression have been diagnosed with bipolar disorder and have been unable to respond to a single treatment attempt. In embodiments, a patient to be treated has not responded to an adequate dose and duration of at least two and no more than five antidepressant medications for the current depressive episode.

In embodiments, the disorder can be diagnosed in accordance with the Diagnostic and Statistical Manual of Mental Disorders-Fifth Edition (DSM-5) published by the American Psychiatric Association. In embodiments, the patient has moderate or severe major depressive disorder as indicated by a Montgomery-Åsberg Depression Rating Scale (MADRS) total score of 20 or more or by a Hamilton Depression Rating Scale (HAM-D) score of 17 or more. In embodiments, the patient has severe major depressive disorder as indicated by a MADRS total score of 35 or more or by a HAM-D score of 25 or more. In embodiments, the patient has a treatment-resistant form of major depressive disorder as indicated by a MADRS score of about 20 or more. In embodiments, the patient that is treated with the methods of the present disclosure has a MADRS score that is at least about 20, at least about 25, at least about 30, at least about 35, at least about 40, at least about 45, at least about 50, at least about 55, about 20− about 25, about 25−about 30, about 30− about 35, about 35− about 40, about 40− about 45, about 45− about 50, about 50− about 55, about 55− about 60, about 25− about 35, about 35− about 45, about 45− about 60, about 25− about 40, or about 40− about 60.

Measures of treatment effect by the methods of the present disclosure include, but are not limited to: Montgomery-Asberg Depression Rating Scale (MADRS), Quality of Life Enjoyment and Satisfaction Questionnaire Short Form, Range of Impaired Functioning Tool, Sheehan Disability Scale, Patient Rated Inventory of Side Effects (PRISE), Columbia-Suicide Severity Rating Scale (C-SSRS), Quick Inventory of Depressive Symptomatology, Self Report (QIDS-SR), Clinical Global Impression (CGI) scale, Massachusetts General Hospital Cognitive and Physical Functioning Questionnaire (CPFQ), 17-item Hamilton Rating Scale for Depression (HAM-D17), Massachusetts General Hospital Antidepressant Treatment Response Questionnaire (MGH ATRQ), 16-item Quick Inventory of Depressive Symptomatology-Self Report (QIDS-SR16), Sheehan Disability Scale (SDS), Clinical Global Impression of Severity of Illness (CGI-S), Clinical Global Impression of Change (CGI-C), EuroQOL 5 Dimension 5 Level (EQ-5D-5L), Patient Global Impression of Change (PGIC), 7-item Generalized Anxiety Disorder (GAD-7), Clinical Global Impressions-Improvement (CGI-I). Sheehan Disability Scale (SDS), 16-item Quick Inventory of Depressive Symptomatology-Self Report (QIDS-SR16), Hamilton Anxiety Scale (HAM-A), Massachusetts General Hospital Cognitive and Physical Functioning Questionnaire (CPFQ), CPFQ-Cognitive subscales (Items 4 to 7), Brief Psychiatric Rating Scale (BPRS), Digit Symbol Substitution Test (DSST), Rey Auditory Verbal Learning Task (RAVLT), Trail Making Test (TMT), Stroop Colour Naming Test (STROOP), Simple Reaction Time (SRT), Choice Reaction Time (CRT). Other procedures, methodologies, or techniques useful for assessing treatment effects known to those skilled in the art may alternatively be used.

In embodiments, the methods of the present disclosure provides a reduction in the patient's MADRS total score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provides a reduction in the patient's MADRS total score by at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, or more, compared to prior to administration. In embodiments, the methods of the present disclosure provides remission as assessed by patient's MADRS total score equal to or less than 10.

In embodiments, the methods of the present disclosure provide a reduction in the patient's HAM-D score by at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the patient's HAM-D score by at least about 5, at least about 6, at least about 7, at least about 8, at least about 9, at least about 10, at least about 11, at least about 12, at least about 13, at least about 14, at least about 15, or more, compared to prior to administration. In embodiments, the methods of the present disclosure provides remission as assessed by patient's HAM-D score equal to or less than 7.

In embodiments, the clinical response, as assessed by at least 50% improvement of the MADRS or HAM-D score, compared to the respective score prior to administration, persists until at least about 6 days, at least about 14 days, at least about 28 days, after the last administration of the composition described herein.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts or attempts, and/or self- deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

In embodiments, the methods provided herein reduce at least one sign or symptom of depressive disorder.

In embodiments, the methods provided herein reduce at least one sign or symptom of major depressive disorder.

### NUMBERED EMBODIMENTS OF THE DISCLOSURE

In addition to the disclosure above, the Examples below, and the appended claims, the disclosure sets forth the following numbered embodiments.

1. A method of treating treatment resistant depression in a patient in need thereof, the method comprising administering a mucoadhesive composition comprising N, N-dimethyltryptamine (DMT) or a pharmaceutically acceptable salt thereof,
wherein the administration provides a DMT Tₘₐₓ of about 0.3 h to about 2 h and a DMT Cₘₐₓ of about 5 ng/mL to about 80 ng/mL following administration.

2. A method of treating treatment resistant depression in a patient in need thereof, the method comprising administering a mucoadhesive composition comprising N, N-dimethyltryptamine (DMT) or a pharmaceutically acceptable salt thereof,
wherein the administration provides a DMT Tₘₐₓ of about 0.3 h to about 2 h and a DMT AUCₗₐₛₜ of about 3 ng·h/mL to about 70 ng. h/mL following administration.

3. The method of embodiment 1 or 2, wherein the administration provides a DMT Tₘₐₓ of about 0.5 h to about 1.5 h following administration.

4. The method of embodiment 1 or 2, wherein the administration provides a DMT Tₘₐₓ of about 0.6 h to about 0.8 h following administration.

5. The method of embodiment 2, wherein the administration provides a DMT Cₘₐₓ of DMT of about 5 ng/mL to about 80 ng/mL following administration.

6. The method of any one of embodiments 1-5, wherein the administration provides a DMT Cₘₐₓ of about 5 ng/mL to about 10 ng/mL following administration.

7. The method of any one of embodiments 1-5, wherein the administration provides a DMT Cₘₐₓ of about 10 ng/mL to about 30 ng/mL following administration.

8. The method of any one of embodiments 1-5, wherein the administration provides a DMT Cₘₐₓ of about 20 ng/mL to about 50 ng/mL following administration.

9. The method of any one of embodiments 1-5, wherein the administration provides a DMT Cₘₐₓ of about 25 ng/mL to about 80 ng/mL following administration.

10. The method of embodiment 1, wherein the administration provides a DMT AUCₗₐₛₜ of about 3 ng·h/mL to about 70 ng·h/mL following administration.

11. The method of any one of embodiments 1-10, wherein the administration provides a DMT AUCₗₐₛₜ of about 3 ng·h/mL to about 9 ng·h/mL following administration.

12. The method of any one of embodiments 1-10, wherein the administration provides a DMT AUCₗₐₛₜ of about 8 ng·h/mL to about 30 ng·h/mL following administration.

13. The method of any one of embodiments 1-10, wherein the administration provides a DMT AUCₗₐₛₜ of about 15 ng·h/mL to about 50 ng·h/mL following administration.

14. The method of any one of embodiments 1-10, wherein the administration provides a DMT AUCₗₐₛₜ of about 25 ng·h/mL to about 70 ng·h/mL following administration.

15. The method of any one of embodiments 1-14, wherein about 20 mg to about 200 mg of DMT free base or an equivalent dose of the pharmaceutically acceptable salt thereof is administered to the patient.

16. The method of any one of embodiments 1-15, wherein about 20 mg to about 60 mg of DMT free base or an equivalent dose of the pharmaceutically acceptable salt thereof is administered to the patient.

17. The method of any one of embodiments 1-15, wherein about 60 mg to about 120 mg of DMT free base or an equivalent dose of the pharmaceutically acceptable salt thereof is administered to the patient.

18. The method of any one of embodiments 1-15, wherein about 120 mg to about 160 mg of DMT free base or an equivalent dose of the pharmaceutically acceptable salt thereof is administered to the patient.

19. The method of any one of embodiments 1-15, wherein about 60 mg of DMT free base or an equivalent dose of the pharmaceutically acceptable salt thereof is administered to the patient.

20. The method of any one of embodiments 1-15, wherein about 120 mg of DMT free base or an equivalent dose of the pharmaceutically acceptable salt thereof is administered to the patient.

21. The method of any one of embodiments 1-20, wherein the composition is administered buccally.

22. The method of any one of embodiments 1-20, wherein the composition is administered through a transmucosal application.

23. The method of embodiment 22, wherein the transmucosal application is a buccal, sublingual, gingival, on the tongue, nasal, vaginal, or rectal application.

24. The method of any one of embodiments 1-23, wherein the composition is administered once about every 2 weeks to about 6 months.

25. The method of any one of embodiments 1-24, wherein the administration provides a reduction in the patient's Montgomery-Asberg Depression Rating Scale (MADRS) total score by at least about 50% compared to prior to the administration.

26. The method of any one of embodiments 1-24, wherein the administration provides a reduction in the patient's MADRS total score by at least about 6 compared to prior to the administration.

27. The method of any one of embodiments 1-24, wherein the administration provides remission according to the patient's MADRS total score after the administration.

28. The method of any one of embodiments 1-27, wherein the patient is at least 18 years old.

29. The method of any one of embodiments 1-28, wherein the patient has a diagnosis of moderate or severe major depressive disorder (MDD) without psychotic features and is currently experiencing a major depressive episode.

30. The method of embodiment 29, wherein the patient has the onset of the first episode of MDD before age 55.

31. The method of embodiment 29 or 30, wherein the patient has not responded to an adequate dose and duration of at least two and no more than five antidepressant medications for the current depressive episode.

32. The method of any one of embodiments 29-31, wherein the patient has a MADRS total score of at least about 20 prior to the administration.

33. A method of treating treatment resistant depression (TRD) by administering N, N-dimethyltryptamine (DMT) or a pharmaceutically acceptable salt thereof to a patient in need thereof, the method comprising:
a) administering to the patient an induction regimen comprising a DMT induction dose; and
b) thereafter administering to the patient a maintenance regimen comprising a DMT maintenance dose.

34. A method of treating treatment resistant depression (TRD) by administering DMT or a pharmaceutically acceptable salt thereof to a patient in need thereof, the method comprising:
a) administering to the patient an induction regimen comprising a DMT induction dose, wherein the induction regimen provides remission of the patient's TRD; and
b) thereafter administering to the patient a maintenance regimen comprising a DMT maintenance dose.

35. The method of embodiment 33 or 34, wherein the DMT is administered in an oral film composition.

36. The method of any one of embodiments 33-35, wherein the induction regimen comprises one DMT dose.

37. The method of any one of embodiments 33-35, wherein the induction regimen comprises multiple DMT doses.

38. The method of embodiment 37, wherein the DMT induction doses are administered at an interval of between about 1 week and about 12 weeks between the doses.

39. The method of embodiment 37 or 38, wherein the DMT induction doses are administered between once a week and once every 12 weeks.

40. The method of embodiment 39, wherein the induction doses are administered once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks.

41. The method of any one of embodiments 33-40, wherein the induction regimen is about 1 week to about 24 weeks.

42. The method of embodiment 39, wherein the induction regimen is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, or about 24 weeks.

43. The method of any one of embodiments 33-42, wherein the induction regimen comprises about 1 to about 10 DMT induction doses.

44. The method of embodiment 43, wherein the induction regimen comprises about 1 to about 10 DMT induction doses once every 2 weeks.

45. The method of embodiment 44, wherein the induction regimen comprises 2 DMT induction doses once every 2 weeks.

46. The method of any one of embodiments 33-45, wherein the DMT induction dose is from about 20 mg to about 200 mg of DMT free base or an equivalent amount of the pharmaceutically acceptable salt thereof.

47. The method of embodiment 46, wherein the DMT induction dose is about 120 mg of DMT free base or an equivalent amount of the pharmaceutically acceptable salt thereof.

48. The method of any one of embodiments 33-47, wherein the DMT induction regimen comprises 2 induction doses each comprising about 120 mg of DMT free base or an equivalent amount of the pharmaceutically acceptable salt thereof.

49. The method of any one of embodiments 33, 35-48, wherein the induction regimen provides remission of the patient's TRD.

50. The method of any one of embodiments 33-49, wherein the induction regimen provides a reduction in the patient's MADRS total score by at least about 20%, at least about 30%, at least about 40%, or at least about 50% compared to prior to the administration.

51. The method of embodiment 50, wherein the induction regimen provides a reduction in the patient's MADRS total score by at least about 50% compared to prior to the administration.

52. The method of any one of embodiments 33-49, wherein the induction regimen provides a reduction in the patient's MADRS total score by at least about 6 compared to prior to the administration.

53. The method of any one of embodiments 33-52, wherein the maintenance regimen comprises one DMT maintenance dose.

54. The method of any one of embodiments 33-52, wherein the maintenance regimen comprises multiple DMT maintenance doses.

55. The method of any one of embodiments 54, wherein the DMT maintenance doses are administered at an interval of between about 1 week and about 12 weeks between the doses.

56. The method of embodiment 55, wherein the DMT maintenance doses are administered between once a week and once every 12 weeks.

57. The method of embodiment 56, wherein the DMT maintenance doses are administered once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks.

58. The method of embodiment 54, wherein the DMT maintenance doses are administered once a month, once every 2 months, once every 3 months, or once every 4 months.

59. The method of any one of embodiments 33-58, wherein the maintenance regimen is about 1 week to about 96 weeks.

60. The method of embodiment 33-58, wherein the maintenance regimen is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 53 week, about 54 weeks, about 55 weeks, about 56 weeks, about 57 weeks, about 58 weeks, about 59 weeks, about 60 weeks, about 61 weeks, about 62 weeks, about 63 weeks, about 64 weeks, about 65 weeks, about 66 weeks, about 67 weeks, about 68 weeks, about 69 weeks, about 70 weeks, about 71 weeks, about 72 weeks, about 73 weeks, about 74 weeks, about 75 weeks, about 76 weeks, about 77 weeks, about 78 weeks, about 79 weeks, about 80 weeks, about 81 weeks, about 82 weeks, about 83 weeks, about 84 weeks, about 85 weeks, about 86 weeks, about 87 weeks, about 88 weeks, about 89 weeks, about 90 weeks, about 91 weeks, about 92 weeks, about 93 weeks, about 94 weeks, about 95 weeks, about 96 weeks, about 97 weeks, about 98 weeks, or more weeks.

61. The method of any one of embodiments 33-58, wherein the maintenance regimen is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, or more months.

62. The method of any one of embodiments 33-61, wherein the maintenance regimen comprises about 1 to about 20 DMT maintenance doses.

63. The method of embodiment 62, wherein the maintenance regimen comprises 1 to about 20 once a week DMT maintenance doses.

64. The method of embodiment 63, wherein the maintenance regimen comprises about 6 once a week DMT maintenance doses.

65. The method of embodiment 62, wherein the maintenance regimen comprises about 1 to about 20 once a month DMT maintenance doses.

66. The method of embodiment 65, wherein the maintenance regimen comprises about 3 once a month DMT maintenance doses.

67. The method of any one of embodiments 33-58, wherein the maintenance regimen is administered chronically to maintain remission of the patient's TRD.

68. The method of any one of embodiments 33-67, wherein the DMT maintenance dose is from about 10 mg to about 200 mg of DMT free base or an equivalent amount of the pharmaceutically acceptable salt thereof.

69. The method of embodiment 68, wherein the DMT maintenance dose is about 60 mg of DMT free base or an equivalent amount of the pharmaceutically acceptable salt thereof.

70. The method of embodiment 68, wherein the DMT maintenance dose is about 120 mg of DMT free base or an equivalent amount of the pharmaceutically acceptable salt thereof.

71. The method of any one of embodiments 33-70, wherein the DMT maintenance dose is about 10%, about 20%, about 30%, about 40%, about 50%, about 60%, about 70%, about 80%, about 90%, or about 100% of the DMT induction dose.

72. The method of any one of embodiments 33-71, wherein the first DMT maintenance dose is administered about 1 week to about 24 weeks after the last DMT induction dose.

73. The method of any one of embodiments 33-72, wherein the first DMT maintenance dose is administered about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 week, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after the last DMT induction dose.

74. The method of any one of embodiments 33-73, wherein the maintenance regimen maintains remission of the patient's TRD.

75. The method of embodiment 33, wherein the method comprises:
a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and
b) thereafter administering to the patient a DMT maintenance dose of about 120 mg of DMT free base or an equivalent amount of the pharmaceutically acceptable salt thereof.

76. The method of embodiment 33, wherein the method comprises:
a) administering to the patient 2 DMT induction doses each comprising about 120 mg of DMT free base or an equivalent amount of the pharmaceutically acceptable salt thereof, wherein the DMT induction doses are administered once every 2 weeks; and
b) thereafter administering to the patient a DMT maintenance dose of about 120 mg of DMT free base or an equivalent amount of the pharmaceutically acceptable salt thereof.

77. The method of embodiment 75, wherein the maintenance dose is administered about 12 weeks after the last induction dose.

78. The method of embodiment 33, wherein the method comprises:
a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and
b) thereafter administering to the patient a DMT maintenance dose of about 60 mg of DMT free base or an equivalent amount of the pharmaceutically acceptable salt thereof.

79. The method of embodiment 33, wherein the method comprises:
a) administering to the patient 2 DMT induction doses each comprising about 120 mg of DMT free base or an equivalent amount of the pharmaceutically acceptable salt thereof, wherein the DMT induction doses are administered once every 2 weeks; and
b) thereafter administering to the patient a DMT maintenance dose of about 60 mg of DMT free base or an equivalent amount of the pharmaceutically acceptable salt thereof.

80. The method of embodiment 78, wherein the maintenance dose is administered about 12 weeks after the last induction dose.

81. The method of embodiment 33, wherein the method comprises:
a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and
b) thereafter administering to the patient about 1 to about 20 once a week DMT maintenance doses each comprising about 60 mg to about 120 mg of DMT free base or an equivalent amount of the pharmaceutically acceptable salt thereof.

82. The method of embodiment 33, wherein the method comprises:
a) administering to the patient 1 to about 10 DMT induction doses each comprising about 120 mg of DMT free base or an equivalent amount of the pharmaceutically acceptable salt thereof, wherein the DMT induction doses are administered between once a week to once every 4 weeks; and
b) thereafter administering to the patient 1 to about 20 once a month DMT maintenance doses each comprising about 60 mg to about 120 mg of DMT free base or an equivalent amount of the pharmaceutically acceptable salt thereof.

83. The method of embodiment 33, wherein the method comprises:
a) administering to the patient a DMT induction dose once every 4 weeks; and
b) thereafter administering to the patient a DMT maintenance dose once a month.

84. The method of embodiment 33, wherein the method comprises:
a) administering to the patient 2 DMT induction doses once every 4 weeks; and
b) thereafter administering to the patient 5 DMT maintenance doses once a month.

85. The method of any one of embodiments 33-84, wherein the patient is at least 18 years old.

86. The method of any one of embodiments 33-85, wherein the patient has a diagnosis of moderate or severe major depressive disorder (MDD) without psychotic features and is currently experiencing a major depressive episode.

87. The method of embodiment 86, wherein the patient has the onset of the first episode of MDD before age 55.

88. The method of embodiment 86 or 87, wherein the patient has not responded to an adequate dose and duration of at least two and no more than five antidepressant medications for the current depressive episode.

89. The method of any one of embodiments 86-88, wherein the patient has a MADRS total score of at least about 20 prior to the administration.

### EXAMPLES

The following examples are provided to further illustrate the embodiments of the present disclosure but are not intended to limit the scope of the claims. While they are typical of those that might be used, other procedures, methodologies, or techniques known to those skilled in the art may alternatively be used.

### Example 1. Adminstration of DMT Buccal Transmucosal Composition

A Phase 1b, single-center, open-label, two-part study of DMT administered via intravenously administration (DMT-IV), via buccal administration using the oral thin film (OTF) strips (DMT-BU), and via subcutaneous administration (DMT-SC) in healthy volunteers. The primary objective was to assess the plasma pharmacokinetics (PK) and urine PK characteristics of DMT and DMT 2 predominant metabolites (N,N-dimethyltryptamine-N-oxide [DMT-NO] and indole-3-acetic acid [IAA]) when administered as an IV infusion, BU via an OTF and via SC injection/infusion.

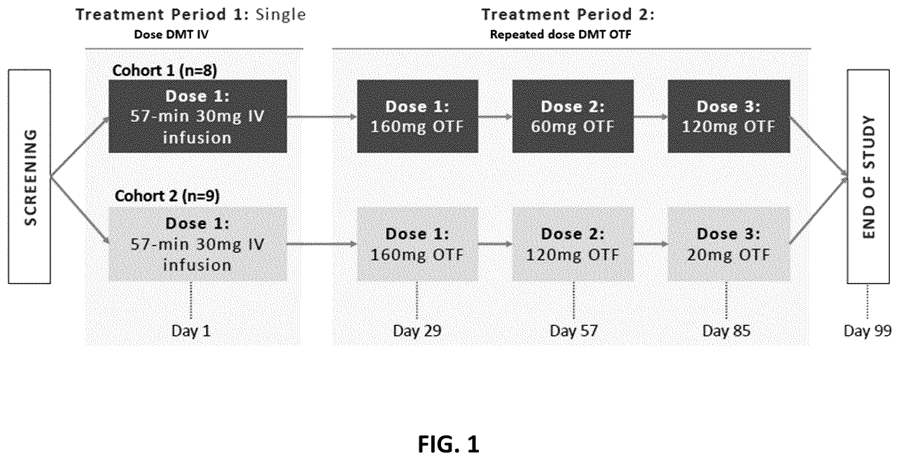

Part A had two cohorts of 8 participants per cohort. Both cohorts in Part A received a total of 4 administrations of DMT, with a 28-day washout between administrations. Following an up to 28-day screening period, all participants in Part A received a single 30 mg IV infusion of DMT-IV administered over 57-minutes (Treatment Period 1), followed by 3 doses of DMT-BU (Treatment Period 2), and a safety follow-up visit 14 days after the last dose of Investigational medicinal product (IMP). The two cohorts in Part A received a starting dose of 160 mg of DMT-BU. Both cohorts then received a second dose of DMT-BU at either 60 mg or 120 mg and a third dose of DMT-BU at 120 mg or 20 mg, with each cohort receiving a different dose of DMT-BU. The study design for Part A is presented in FIG. 1.

The treatment with repeated doses of DMT OTF was safe and tolerated by study participants. There were no serious adverse events (SAEs) or severe adverse events (AEs). Optimized DMT OTF demonstrated good tolerability with all adverse events classified as either mild or moderate, and most resolving on the day of dosing.

Serial blood samples were collected for the determination of concentrations of DMT and its predominant metabolites (DMT-NO and IAA). PK sampling timepoints are provided in Table 8.

TABLE 8
Pharmacokinetic Sampling Timepoints

| Sample Type | Dosing Day | Time Postdose |
|---|---|---|
| Plasma | 1, 29, | 10, 20, 30, 40, 50, 57, 75, 90, 105, 120 |
| | 57, 85 | (each ± 2 minutes), 150, 180, 240, 360, |
| | | and 480 minutes (each ± 10 minutes) |

The plasma PK parameters measured in this study are provided in Table 9.

TABLE 9
Pharmacokinetic Parameters

| Parameter | Definition |
|---|---|
| Cₘₐₓ | Maximum observed plasma concentration. If the same Cₘₐₓ concentration occurs at |
| | different time points, Tₘₐₓ is assigned to the first occurrence of Cₘₐₓ. |
| Tₘₐₓ | Time to maximum concentration. |
| AUCₗₐₛₜ | Area under the drug concentration-time curve, from time zero (time of dosing) to the |
| | last measurable concentration using the ‘Linear Up and Log Down’ method. |
| AUC_{0-inf} | The area under the plasma drug concentration-time curve from time 0 (time of |
| | dosing) extrapolated to infinity. AUC_{0-inf} is calculated as the sum of AUC₀₋ₜ plus the |
| | ratio of the last measurable plasma concentration (Cₗₐₛₜ) to the elimination rate |
| | constant (λ_{z}), calculated by the ‘Linear Up and Log Down’ method. |
| Half-life (t_{1/2}) | Apparent terminal half-life, determined using the following formula: |
| | t_{1/2} = ln(2)/λ_{z} |
| CL/F or CL | Apparent total body clearance, determined using the following formula: |
| | CL/F or CL = Dose in mg/AUC_{0-inf} |
| | (CL for IV infusion and CL/F for buccal route). |
| Vz/F or Vz | Apparent terminal volume of distribution, determined using the following formula: |
| | V_{z}/F or Vz = Dose in mg/λ_{z} × AUC_{0-inf} |
| | (Vz for IV infusion and Vz/F for buccal route). |
| Cₘₐₓ/Dose | Dose-normalized Cₘₐₓ, calculated as Cₘₐₓ divided by the dose in mg administered. |
| AUCₗₐₛₜ/Dose | Dose-normalized AUC₀₋ₜ, calculated as AUC₀₋ₜ divided by dose in mg administered. |
| SIRS Max | Maximum SIRS rating over the course of treatment |
| Metabolite Ratio | AUCₗₐₛₜ of the metabolite/AUCₗₐₛₜ of DMT |
| (AUClastₘₑₜ/ | |
| AUClast_{DMT}) | |

Abbreviations: BU = buccal; IV = intravenous; PK = pharmacokinetic.

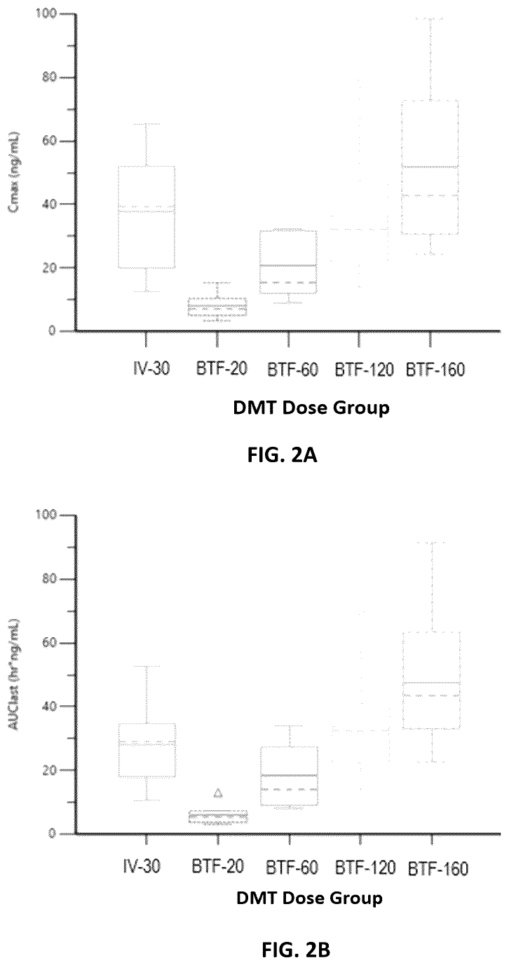

The PK results were conducted using an NCA with nominal times collapsing across cohort. The preliminary PK parameters for DMT, DMT-NO and IAA are shown in Table 10 and the Cₘₐₓ and AUCₗₐₛₜ box plots for DMT by dose group are shown in FIGS. 2A and 2B.

TABLE 10
Summary of Plasma Pharmacokinetic Parameter Results

| Treatment | | DMT | | | DMT-NO | | | IAA^{a} | | |
|---|---|---|---|---|---|---|---|---|---|---|
| Group | Parameter | n | Value^{b} | | n | Value^{b} | | n | Value^{b} | |
| DMT-IV | Cₘₐₓ (ng/mL) | 16 | 37.7 | (16.2) | 16 | 0.80 | (0.2) | 16 | 2283.3 | (379.4) |
| 30 mg | Tₘₐₓ (hr) | 16 | 0.83 | (0.33, 0.95) | 16 | 0.95 | (0.95, 1.25) | 16 | 1.25 | (0.95, 1.75) |
| (0.53 mg | AUCₗₐₛₜ | 16 | 28.1 | (10.9) | 16 | 1.20 | (0.3) | 16 | 9075.0 | (2443.6) |
| per | t_{1/2} (hr) | 16 | 0.32 | (0.10) | 16 | 1.04 | (0.20) | 16 | 3.36 | (0.87) |
| minute) | MRAUC^{c} | n/a | | | 16 | 0.05 | (0.02) | 16 | 398.8 | (279.1) |
| (n = 17) | | | | | | | | | | |
| DMT-BU | Cₘₐₓ (ng/mL) | 16 | 51.90 | (24.5) | 16 | 35.0 | (17.2) | 16 | 10100.6 | (2190.8) |
| 160 mg | Tₘₐₓ (hr) | 16 | 0.67 | (0.33, 1.25) | 16 | 2.00 | (1.25, 3) | 16 | 2.50 | (1.25, 3) |
| (n = 16) | AUCₗₐₛₜ | 16 | 47.6 | (19.8) | 16 | 58.0 | (20.5) | 16 | 38214.0 | (9259.0) |
| | t_{1/2} (hr) | 16 | 0.34 | (0.11) | 16 | 1.40 | (0.15) | 16 | 2.71 | (0.87) |
| | MRAUC^{c} | n/a | | | 16 | 1.5 | (0.8) | 16 | 979.4 | (530.6) |
| DMT-BU | Cₘₐₓ (ng/mL) | 14 | 36.5 | (17.6) | 14 | 20.8 | (9.4) | 14 | 7693.5 | (1672.9) |
| 120 mg | Tₘₐₓ (hr) | 14 | 0.67 | (0.33, 1.5) | 14 | 1.75 | (1.25, 2.5) | 14 | 2.25 | (1.5, 3) |
| (n = 14) | AUCₗₐₛₜ | 14 | 33.9 | (15.7) | 14 | 37.1 | (11.6) | 14 | 30117.6 | (7459.8) |
| | t_{1/2} (hr) | 14 | 0.32 | (0.06) | 14 | 1.45 | (0.18) | 14 | 3.10 | (1.11) |
| | MRAUC^{c} | n/a | | | 14 | 1.4 | (0.8) | 14 | 1041.7 | (444.9) |
| DMT-BU | Cₘₐₓ (ng/mL) | 7 | 20.8 | (10.1) | 7 | 6.7 | (2.0) | 7 | 3882.6 | (565.7) |
| 60 mg | Tₘₐₓ (hr) | 7 | 0.67 | (0.33, 0.83) | 7 | 1.5 | (1.25, 2.5) | 7 | 2.0 | (1.5, 3) |
| (n = 7) | AUCₗₐₛₜ | 7 | 18.6 | (10.4) | 7 | 12.40 | (2.9) | 7 | 15100.9 | (3945.7) |
| | t_{1/2} (hr) | 7 | 0.33 | (0.12) | 7 | 1.50 | (0.2) | 7 | 2.8 | (1.0) |
| | MRAUC^{c} | n/a | | | 7 | 0.8 | (0.4) | 7 | 994.1 | (405.0) |
| DMT-BU | Cₘₐₓ (ng/mL) | 8 | 7.9 | (3.9) | 8 | 1.70 | (0.60) | 8 | 1828.8 | (262.5) |
| 20 mg | Tₘₐₓ (hr) | 8 | 0.50 | (0.33, 0.67) | 8 | 1.25 | (0.83, 2) | 8 | 1.38 | (0.95, 2) |
| (n = 8) | AUCₗₐₛₜ | 8 | 6.2 | (3.2) | 8 | 2.8 | (0.8) | 8 | 7757.3 | (1875.6) |
| | t_{1/2} (hr) | 8 | 0.33 | (0.09) | 8 | 1.26 | (0.21) | 8 | 4.59 | (1.79) |

| | | | | | | | |
|---|---|---|---|---|---|---|---|
| MRAUC^{c} | n/a | 8 | 0.6 | (0.4) | 8 | 1506.7 | (708.3) |

Abbreviations: AUCₗₐₛₜ = area under the drug concentration-time curve, from time 0 (time of dosing or start of the infusion) to the last measurable concentration; Cₘₐₓ = maximum observed plasma concentration; DMT-NO = N,N-dimethyltryptamine-N-oxide ; IAA = indole-3-acetic acid; MRAUC = metabolic ratio AUC; N/A = not applicable; SD = standard deviation; t_{1/2} = apparent terminal half-life; Tₘₐₓ = time to first maximum observed concentration.
^{a}Baseline correction was done by subtracting the pre-dose level from the concentration at each timepoint. If a Baseline correction resulted in a negative concentration value, the value was set to 0.
^{b}Pharmacokinetic values are reported as mean (SD) for Cₘₐₓ, AUCₗₐₛₜ, t_{1/2}, and MRAUC, and as median (minimum, maximum) for Tₘₐₓ.
^{c}MRAUC was calculated as AUC_{0-inf} metabolite/AUC_{0-inf} parent OR as AUC₀₋ₜ metabolite/AUC₀₋ₜ parent.
Only participants who received a full dose of the treatment to which they were assigned were included in the summaries.

In the buccal dose groups, systemic exposure to DMT, DMT-NO and IAA increased with increasing dose of DMT-BU. DMT Cₘₐₓ and AUCₗₐₛₜ increased with increasing dose in an approximately dose-proportional manner. The Cₘₐₓ and AUCₗₐₛₜ of DMT from the 57-min IV slow infusion was most closely matched by the Cₘₐₓ and AUCₗₐₛₜ of the 120 mg dose of DMT-BU. In all dose groups, IAA represented the majority of circulating drug-related material with mean MRAUC values for IAA/DMT ranging from 399 to ˜1507 (Table 10). In contrast, DMT-NO plasma concentrations were far lower than IAA in all cohorts. Mean MRAUC values for DMT NO/DMT in the BTF dose groups ranging from 0.6-1.5. Very little DMT-NO is produced in the IV infusion cohort.

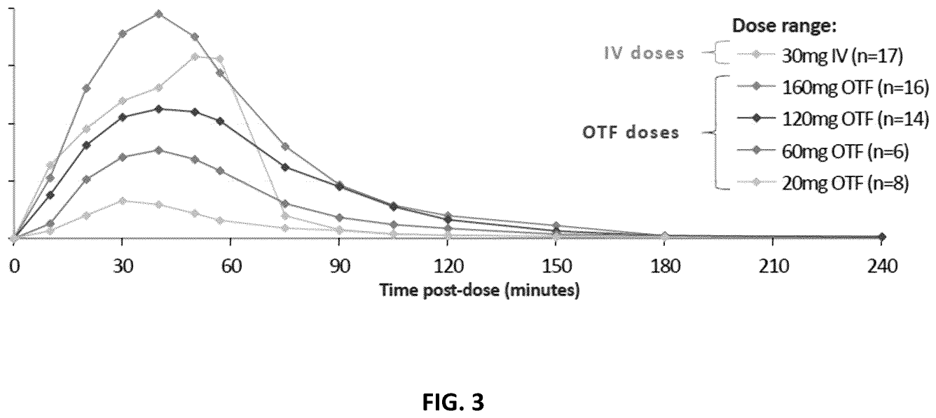

The DMT plasma concentration over time for all cohorts is shown in FIG. 3. DMT OTF rapidly reached peak plasma concentration (Tₘₐₓ) within 30-45 minute.

The pharmacodynamics (PD) parameters measured in this study are provided in Table 11.

TABLE 11
Exploratory PD Assessments

| Assessment | Description |
|---|---|
| Subjective | For participants to verbally estimate intensity of the drug effect in real time |
| Intensity Rating | on a scale of 0 to 10 (range 0 = ‘No effect/the effects have completely worn |
| Scale (SIRS) | off’ to 10 = ‘Greatest intensity imaginable’). SIRS was collected at the PK |
| | sampling timepoints. |
| Challenging | Assessed unpleasant reactions to psychedelic drugs. It covered self-reporting |
| Experience | on acute affective, cognitive, and somatic symptoms pertaining with |
| Questionnaire | subscales of fear, grief, physical distress, insanity, isolation, death, and |
| (CEQ) | paranoia; 26 items on a self-report Likert scale (range 0 to 5: 0 “none/not at |
| | all”, 1 “so slight cannot decide”, 2 “slight”, 3 “moderate”, 4 “strong”, 5 |
| | “extreme (more than ever before in my life)). |
| Visual | Comprises 8 visual analogue scales for participants to indicate how much |
| Analogue | they experience psychedelic effects common to DMT (range: 0 to 100: 1 = |
| Scale (VAS) | “no/not at all” to 100 = “most imaginable”). |
| Mystical | The MEQ is designed to measure four forms of mystical experiences that can |
| Experience | be experienced during a psychedelic experience. The measure includes 30 |
| Questionnaire | items that are rated on a self-report Likert scale (range 0 to 5: 0 = “none/not |
| (MEQ) | at all” to 5 = “extreme [more than any other time in my life and stronger than |
| | 4]”). |

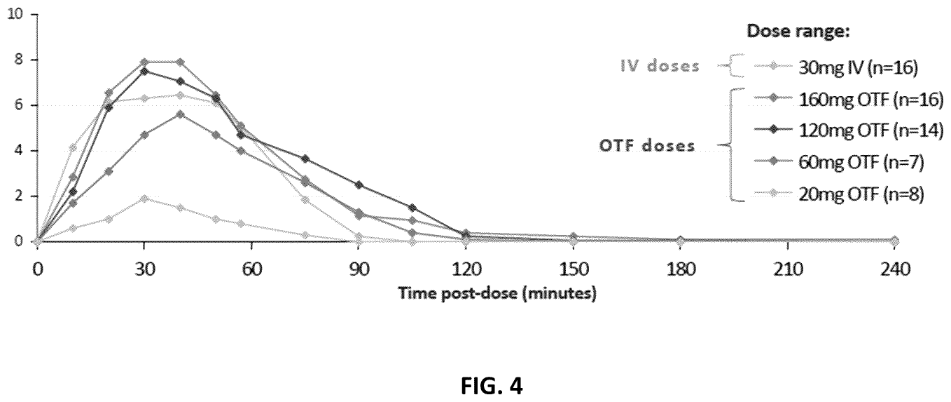

SIRS over time for all cohorts is shown in FIG. 4. DMT OTF groups show dose-dependent effects, with robust patientive effects seen at the 120 mg and 160 mg doses. 13/14 participants in the 120 mg cohort achieved SIRS scores greater than 7. Perceptual effects generally fully resolved within 90-120 minutes. This result indicates that DMT OTF has the potential to offer a predictable dosing model administered around a 2-hour in-clinic treatment paradigm.

Unpleasant reactions to DMT-IV and DMT-BU were assessed using the CEQ. The CEQ included 26 items that participants rated after the psychedelic experience on a scale from 0 to 5 from which subscale scores, categorized by reactions to fear, grief, physical distress, insanity, isolation, death, and paranoia were calculated. Scores for subscales were used to derive the CEQ total score. The summary of CEQ subscale scores and total scores for cohorts 1 and 2 is provided in Table 12.

TABLE 12
CEQ subscale scores and total scores for cohorts 1 and 2

| | | Cohort 1 | | | | Cohort 2 | | | |
|---|---|---|---|---|---|---|---|---|---|
| | | DMT-IV | | | | DMT-IV | | | |
| | | 30 mg | DMT-BU | DMT-BU | DMT-BU | 30 mg | DMT-BU | DMT-BU | DMT-BU |
| | | IV | 160 mg | 60 mg | 120 mg | IV | 160 mg | 120 mg | 20 mg |
| Parameter | | Infusion | OTF | OTF | OTF | Infusion | OTF | OTF | OTF |
| Subscale | Statistic | (N = 8) | (N = 8) | (N = 7) | (N = 6) | (N = 9) | (N = 8) | (N = 8) | (N = 8) |
| Fear | Mean | 5.0 | 9.0 | 1.1 | 0.0 | 28.9 | 43.0 | 11.0 | 1.5 |
| | (SD) | (9.5) | (13.1) | (2.0) | (0.0) | (23.7) | (30.8) | (17.7) | (4.2) |
| Grief | Mean | 1.25 | 9.17 | 3.33 | 0.00 | 11.85 | 24.58 | 12.50 | 0.00 |
| | (SD) | (1.73) | (11.79) | (8.82) | (0.00) | (11.80) | (25.57) | (23.21) | (0.00) |
| Physical | Mean | 17.5 | 19.0 | 1.7 | 3.3 | 28.0 | 26.0 | 23.0 | 5.0 |
| Distress | (SD) | (14.8) | (25.3) | (4.5) | (8.2) | (23.3) | (18.9) | (16.0) | (8.2) |
| Insanity | Mean | 1.67 | 4.17 | 0.00 | 0.00 | 13.33 | 24.17 | 5.83 | 0.00 |
| | (SD) | (4.71) | (11.79) | (0.00) | (0.00) | (17.00) | (19.82) | (8.31) | (0.00) |
| Isolation | Mean | 0.00 | 10.83 | 6.67 | 0.00 | 20.74 | 30.00 | 11.67 | 0.83 |
| | (SD) | (0.00) | (25.68) | (11.55) | (0.00) | (31.88) | (31.67) | (16.23) | (2.36) |
| Death | Mean | 0.0 | 0.0 | 0.0 | 0.0 | 12.2 | 12.5 | 6.3 | 0.0 |
| | (SD) | (0.0) | (0.0) | (0.0) | (0.0) | (17.9) | (20.5) | (11.9) | (0.0) |
| Paranoia | Mean | 0.0 | 0.0 | 0.0 | 0.0 | 1.1 | 2.5 | 1.3 | 0.0 |
| | (SD) | (0.0) | (0.0) | (0.0) | (0.0) | (3.3) | (7.1) | (3.5) | (0.0) |
| CEQ Total | Mean | 4.81 | 9.23 | 2.09 | 0.64 | 18.63 | 26.35 | 12.02 | 1.35 |
| Score | (SD) | (3.90) | (11.11) | (3.19) | (1.57) | (14.70) | (19.39) | (12.92) | (1.78) |

Abbreviations: BU = buccal; CEQ = Challenging Experiences Questionnaire; IV = intravenous (ly); N = number of participants in each group; OTF = oral thin film; SD = standard deviation.
Notes:
DMT-BU = DMT buccal formulation; DMT-IV = DMT intravenous formulation. The CEQ included 26 items that participants rated after the psychedelic experience on a scale from 0 (none/not at all) to 5 (more than ever before in my life) and was completed 4 hours after dosing.

Mystical experience to DMT-IV and DMT-BU were assessed using the MEQ. The MEQ measured 4 forms of mystical experiences (subscales described below) that can be experienced during a psychedelic experience. The measures included 30 items that were rated on a self-report Likert scale (range 0 to 5) from which subscale scores, categorized by reactions to mystical experience, positive mood, transcendence of time/space, and ineffability were calculated. Scores for subscales were used to derive the MEQ total score. The summary of the MEQ subscale scores and total scores for both cohorts is provided in Table 13.

TABLE 13
Summary of MEQ Subscales and Total Scores

| | | Cohort 1 | | | | Cohort 2 | | | |
|---|---|---|---|---|---|---|---|---|---|
| | | DMT-IV | | | | DMT-IV | | | |
| | | 30 mg | DMT-BU | DMT-BU | DMT-BU | 30 mg | DMT-BU | DMT-BU | DMT-BU |
| | | IV | 160 mg | 60 mg | 120 mg | IV | 160 mg | 120 mg | 20 mg |
| Parameter | | Infusion | OTF | OTF | OTF | Infusion | OTF | OTF | OTF |
| Subscale | Statistic | (N = 8) | (N = 8) | (N = 7) | (N = 6) | (N = 9) | (N = 8) | (N = 8) | (N = 8) |
| Mystical | Mean | 1.26 | 1.61 | 0.91 | 1.43 | 1.78 | 1.11 | 1.43 | 0.25 |
| | (SD) | (1.25) | (1.65) | (1.67) | (1.66) | (1.28) | (1.08) | (1.08) | (0.58) |
| Positive mood | Mean | 2.17 | 2.15 | 1.74 | 2.56 | 2.81 | 2.04 | 2.33 | 0.83 |
| | (SD) | (0.75) | (1.41) | (1.46) | (1.71) | (1.39) | (1.38) | (0.98) | (0.77) |
| Transcendence | Mean | 2.52 | 2.94 | 1.17 | 2.17 | 2.89 | 2.31 | 2.19 | 0.65 |
| of time/space | (SD) | (1.42) | (1.64) | (1.50) | (1.85) | (1.48) | (0.99) | (0.72) | (1.06) |
| Ineffability | Mean | 1.83 | 2.92 | 1.24 | 2.39 | 3.30 | 2.83 | 3.08 | 0.58 |
| | (SD) | (1.54) | (1.51) | (1.69) | (1.74) | (1.59) | (1.22) | (1.34) | (0.92) |
| MEQ Total | Mean | 1.75 | 2.11 | 1.16 | 1.90 | 2.36 | 1.71 | 1.93 | 0.48 |
| Score | (SD) | (1.07) | (1.28) | (1.55) | (1.60) | (1.08) | (0.94) | (0.83) | (0.66) |

Abbreviations: BU = buccal; IV = intravenous (ly); MEQ = Mystical Experiences Questionnaire; N = number of participants in each group; OTF = oral thin film; SD = standard deviation.
Notes:
DMT-BU = DMT buccal formulation; DMT-IV = DMT intravenous formulation. The MEQ included 30 items that were rated from 0 (none/not at all) to 5 (extreme [more than any other time in my life and stronger than 4]) and is completed 4 hours after dosing

Subjective experiences to DMT-IV and DMT-BU were assessed using an 8-item VAS. In this assessment, participants were asked to self-report the degree to which they experienced 8 subjective experiences that were unique to the psychedelic effects of DMT on a VAS (range 0 to 100-mm line corresponding to 1 = “no/not at all” to 100 = “most imaginable”). For analysis purposes, the individual scores were converted to a 0 to 1.0 scale by dividing each response by 100. The converted VAS scores were summarized for each question descriptively by treatment group and dose, and individual VAS scores were presented in the by-participant data listings. The summary of the 8-item VAS scores for both cohorts is provided in Table 14.

TABLE 14
Summary of the 8-item VAS scores

| | | Cohort 1 | | | | Cohort 2 | | | |
|---|---|---|---|---|---|---|---|---|---|
| | | DMT-IV | | | | DMT-IV | | | |
| | | 30 mg | DMT-BU | DMT-BU | DMT-BU | 30 mg | DMT-BU | DMT-BU | DMT-BU |
| | | IV | 160 mg | 60 mg | 120 mg | IV | 160 mg | 120 mg | 20 mg |
| Parameter | | Infusion | OTF | OTF | OTF | Infusion | OTF | OTF | OTF |
| Item | Statistic | (N = 8) | (N = 8) | (N = 7) | (N = 6) | (N = 9) | (N = 8) | (N = 8) | (N = 8) |
| Experienced a | Mean | 0.650 | 0.643 | 0.206 | 0.365 | 0.660 | 0.474 | 0.243 | 0.116 |
| different reality or | (SD) | (0.318) | (0.273) | (0.311) | (0.392) | (0.400) | (0.393) | (0.247) | (0.233) |
| dimension | | | | | | | | | |
| Experienced the | Mean | 0.276 | 0.334 | 0.160 | 0.338 | 0.334 | 0.395 | 0.208 | 0.018 |
| presence of | (SD) | (0.344) | (0.301) | (0.206) | (0.365) | (0.353) | (0.404) | (0.291) | (0.012) |
| another conscious | | | | | | | | | |
| being/lifeform | | | | | | | | | |
| Experienced | Mean | 0.600 | 0.565 | 0.256 | 0.495 | 0.591 | 0.614 | 0.408 | 0.024 |
| elaborate/complex | (SD) | (0.375) | (0.386) | (0.335) | (0.373) | (0.446) | (0.419) | (0.311) | (0.015) |
| visual images | | | | | | | | | |
| Saw geometric | Mean | 0.595 | 0.556 | 0.227 | 0.460 | 0.599 | 0.616 | 0.365 | 0.019 |
| patterns | (SD) | (0.398) | (0.434) | (0.287) | (0.424) | (0.478) | (0.397) | (0.282) | (0.010) |
| Experience felt | Mean | 0.433 | 0.394 | 0.159 | 0.305 | 0.219 | 0.215 | 0.195 | 0.099 |
| more ‘real’ than | (SD) | (0.378) | (0.372) | (0.318) | (0.354) | (0.234) | (0.206) | (0.142) | (0.227) |
| this reality | | | | | | | | | |
| Felt I was | Mean | 0.504 | 0.615 | 0.199 | 0.317 | 0.661 | 0.459 | 0.395 | 0.096 |
| displaced from my | (SD) | (0.328) | (0.404) | (0.291) | (0.414) | (0.303) | (0.411) | (0.299) | (0.126) |
| body | | | | | | | | | |
| Felt completely | Mean | 0.465 | 0.523 | 0.193 | 0.453 | 0.674 | 0.524 | 0.333 | 0.048 |
| immersed in the | (SD) | (0.354) | (0.418) | (0.318) | (0.383) | (0.354) | (0.431) | (0.296) | (0.095) |
| visuals | | | | | | | | | |
| Felt unusual | Mean | 0.441 | 0.331 | 0.173 | 0.270 | 0.769 | 0.651 | 0.500 | 0.095 |
| bodily sensations | (SD) | (0.406) | (0.371) | (0.291) | (0.370) | (0.232) | (0.278) | (0.305) | (0.138) |

Abbreviations: BU = buccal; IV = intravenous (ly); N = number of participants in each group; OTF = oral thin film; SD = standard deviation; VAS = Visual Analogue Scale.
Notes:
DMT-BU = DMT buccal formulation; DMT-IV = DMT intravenous formulation. The 8-item VAS measures 8 subjective experiences that were unique to the psychedelic effects of DMT on scale ranging from 0 (no/not at all) to 100 (most imaginable and was administered 4 hours after dosing). For analysis purposes, the individual scores were converted to a 0 to1.0 scale by dividing each response by 100.

### Example 2. Efficacy, Safety, and Tolerability of Repeated Doses of DMT Buccal Transmucosal Composition in a Phase 2 Clinical Study

A Phase 2, multicenter, double-blind, randomized, placebo-controlled trial is conducted to assess the efficacy, safety, and tolerability of repeated doses of DMT buccal film in participants with treatment resistant depression.

The primary objective is to determine the efficacy of DMT-BU compared with placebo in improving depressive symptoms in participants with TRD 4 weeks after initiating treatment. The endpoint is to assess the change from baseline (prior to administration) in Montgomery-Asberg Depression Rating Scale (MADRS) total score. The secondary objectives are: (1) to assess the efficacy of DMT-BU compared to placebo in improving depressive symptoms in participants with TRD 6-weeks after initiating treatment; (2) to assess the proportion of treatment responders after DMT-BU treatment, compared with placebo, 4 weeks after initiating treatment; (3) to assess the proportion of participants achieving remission after DMT-BU treatment, compared with placebo, 4 weeks after initiating treatment; and (4) to assess the durability of the antidepressant treatment response 12 weeks after receiving two doses of DMT-BU, compared to placebo. The corresponding endpoints include (1) change from Baseline in MADRS total score at Day 43; (2) Proportion of treatment responders (defined as ≥ 50% decrease from Baseline in MADRS total score) at Day 29; (3) Proportion of participants meeting criteria for remission (defined as MADRS total score ≤10) at Day 29; and (4) change from Baseline in MADRS total score at Day 99 pre-dose.

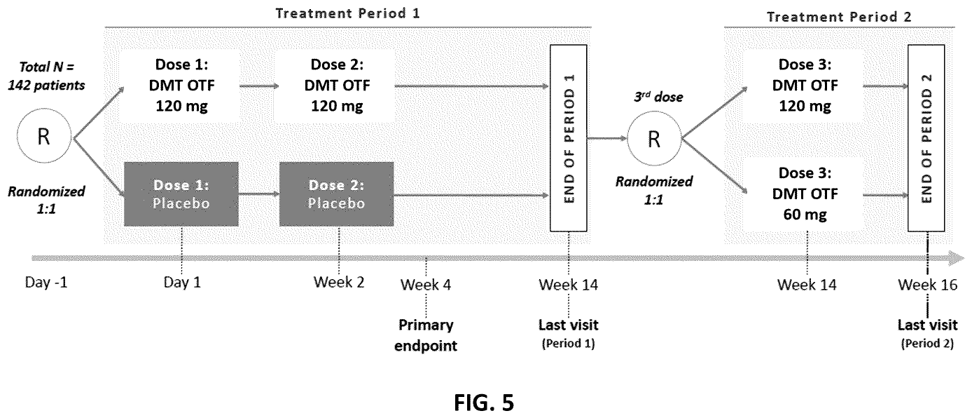

This Phase 2 trial will enroll approximately 142 participants (up to a maximum of 168) with TRD, randomized 1:1 to receive a total of 2 double-blinded administrations of DMT-BU or placebo during the placebo-controlled treatment period. All participants will have their symptoms monitored for 12 weeks (placebo-controlled follow-up period) following the second administration of DMT-BU or placebo. All participants will then be re-randomized 1:1to receive one double-blinded administration of DMT-BU 120 mg or DMT-BU 60 mg (non-placebo-controlled treatment period). The study design for phase 2 trial is presented in FIG. 5.

Participants are eligible to be included in the study only if all the following criteria apply.

### Age

Participant must be at least 18 years of age inclusive at the time of signing the informed consent.

### Type of Participant and Disease Characteristics

Participant has a diagnosis of recurrent moderate or severe MDD without psychotic features or single-episode major depressive disorder (MDD) with duration greater than or equal to 3 months without psychotic features and is currently experiencing a major depressive episode as defined by the Diagnostic and Statistical Manual of Mental Disorders, Fifth Edition Text Revision (DSM-5-TR) and confirmed by the Mini-International Neuropsychiatric Interview (MINI) and SAFER criteria at Screening.

Participant has onset of first episode of MDD occurred before age 55.

Participant has not responded to an adequate dose and duration of at least two and no more than five antidepressant medications for the current depressive episode, as determined by the Massachusetts General Hospital Antidepressant Treatment Response Questionnaire (and confirmed by clinical interview or clinical documentation if available (e.g., pharmacy, hospital, or other health records of previous treatment). Augmentation with an add-on treatment counts as a second treatment, provided it is approved for the adjunctive treatment of MDD in that country.

Participant has a MADRS total score ≥20 at Screening and Day −1.

Participants who are not lactating or pregnant as confirmed by a serum pregnancy test at Screening and negative urine pregnancy test before dosing (applies only to participants of childbearing potential).

Participants are excluded from the study if any of the following criteria apply.

Participant has a current or prior DSM-5-TR diagnosis of a schizophrenia spectrum and other psychotic disorder, substance/medication-induced psychotic disorder, bipolar and related disorder, or any disorder with psychotic features (including MDD with psychotic features), as assessed by medical history and a structured clinical interview (version 7.0.2 MINI).

Participant has a current or prior DSM-5-TR diagnosis of a neurocognitive disorder, intellectual disorder, dissociative disorder, disruptive/impulse-control/conduct disorder, autism spectrum disorder level 2 or 3, or cluster A and B personality disorder, as assessed by medical history and a structured clinical interview (version 7.0.2 MINI). Inclusion of individuals with a diagnosis of autism spectrum disorder level 1, or prior diagnosis of cluster C personality disorder may be considered at the discretion of the investigator if the participant no longer meets criteria for the condition and current functioning and/or subthreshold symptoms will not interfere with MDD treatment or compliance in the study.

Participant has a current DMS-5-TR diagnosis of posttraumatic stress disorder, acute stress disorder, obsessive-compulsive disorder, anorexia nervosa, bulimia nervosa, or any other comorbid psychiatric condition that dominates the clinical presentation and would interfere with experimental treatment.

Participant has a mild, moderate, or severe substance use disorder (drug, alcohol, or tobacco) within the 6 months before Screening and/or history of moderate or severe substance use disorder (drug or alcohol) within the previous 1 year before Screening.

Participant has suicidal ideation with some intent to act within 6 months before Screening or a history of suicidal behavior within the past 1 year before Screening. Suicide risk should be informed by clinical judgement, scoring 17 or higher on the MINI Suicidal Scale at Screening, or endorsing “Yes” on Item 4 (active suicidal ideation with some intent to act, without specific plan) or Item 5 (active suicidal ideation with specific plan and intent) on the CSSRS at Day −1, or MADRS item 10 “suicidal thoughts” score =6 at Screening or Baseline.

1. A method of treating treatment resistant depression (TRD) in a patient in need thereof, the method comprising administering a mucoadhesive composition comprising N, N-dimethyltryptamine (DMT) or a pharmaceutically acceptable salt thereof,
wherein the administration provides a DMT Tₘₐₓ of about 0.3 h to about 2 h and a DMT Cₘₐₓ of about 5 ng/mL to about 80 ng/mL following administration.

2. A method of treating treatment resistant depression (TRD) in a patient in need thereof, the method comprising administering a mucoadhesive composition comprising N, N-dimethyltryptamine (DMT) or a pharmaceutically acceptable salt thereof,
wherein the administration provides a DMT Tₘₐₓ of about 0.3 h to about 2 h and a DMT AUCₗₐₛₜ of about 3 ng·h/mL to about 70 ng·h/mL following administration.

3. The method of claim 1, wherein the administration provides a DMT Tₘₐₓ of about 0.5 h to about 1.5 h following administration.

4. The method of claim 1, wherein the administration provides a DMT Tₘₐₓ of about 0.6 h to about 0.8 h following administration.

5. (canceled)

6. The method of claim 1, wherein the administration provides a DMT Cₘₐₓ of about 5 ng/ml to about 10 ng/mL following administration.

7. The method of claim 1, wherein the administration provides a DMT Cₘₐₓ of about 10 ng/mL to about 30 ng/ml following administration.

8. The method of claim 1, wherein the administration provides a DMT Cₘₐₓ of about 20 ng/ml to about 50 ng/ml following administration.

9. The method of claim 1, wherein the administration provides a DMT Cₘₐₓ of about 25 ng/ml to about 80 ng/ml following administration.

10. The method of claim 1, wherein the administration provides a DMT AUCₗₐₛₜ of about 3 ng·h/mL to about 70 ng·h/mL following administration.

11. (canceled)

12. (canceled)

13. (canceled)

14. (canceled)

15. The method of claim 1, wherein about 20 mg to about 200 mg of DMT free base or an equivalent dose of the pharmaceutically acceptable salt thereof is administered to the patient.

16. (canceled)

17. The method of claim 1, wherein about 60 mg to about 120 mg of DMT free base or an equivalent dose of the pharmaceutically acceptable salt thereof is administered to the patient.

18. (canceled)

19. The method of claim 1, wherein about 60 mg of DMT free base or an equivalent dose of the pharmaceutically acceptable salt thereof is administered to the patient.

20. The method of claim 1, wherein about 120 mg of DMT free base or an equivalent dose of the pharmaceutically acceptable salt thereof is administered to the patient.

21. The method of claim 1, wherein the composition is administered buccally.

22. (canceled)

23. (canceled)

24. The method of claim 1, wherein the composition is administered once about every about 2 weeks to about 6 months.

25. The method of claim 1, wherein the administration provides a reduction in the patient's Montgomery-Asberg Depression Rating Scale (MADRS) total score by at least about 50% compared to prior to the administration.

26. (canceled)

27. (canceled)

28. The method of claim 1, wherein the patient is at least 18 years old.

29. The method of claim 1, wherein the patient has a diagnosis of moderate or severe major depressive disorder (MDD) without psychotic features and is currently experiencing a major depressive episode.

30. (canceled)

31. (canceled)

32. (canceled)

33. A method of treating treatment resistant depression (TRD) by administering N, N-dimethyltryptamine (DMT) or a pharmaceutically acceptable salt thereof to a patient in need thereof, the method comprising:
a) administering to the patient an induction regimen comprising a DMT induction dose; and
b) thereafter administering to the patient a maintenance regimen comprising a DMT maintenance dose.

34. A method of treating treatment resistant depression (TRD) by administering DMT or a pharmaceutically acceptable salt thereof to a patient in need thereof, the method comprising:
a) administering to the patient an induction regimen comprising a DMT induction dose, wherein the induction regimen provides remission of the patient's TRD; and
b) thereafter administering to the patient a maintenance regimen comprising a DMT maintenance dose.

35.-89. (canceled)
